(19) United States
(12) Patent Application Publication
(10) Pub. No.: US 2025/0114476 A1
(43) Pub. Date: Apr. 10, 2025

(54) POLY(ALKYLOXAZOLINE)-LIPID CONJUGATES AND LIPID PARTICLES CONTAINING SAME

(71) Applicant: Genevant Sciences GmbH, Basel (CH)

(72) Inventors: James HEYES, Vancouver (CA); Kieu Mong LAM, Richmond (CA); Richard J. HOLLAND, Burnaby (CA)

(73) Assignee: Genevant Sciences GmbH, Basel (CH)

(21) Appl. No.: 18/832,438

(22) PCT Filed: Jan. 30, 2023
(86) PCT No.: PCT/IB2023/050796

§ 371 Date: Jul. 23, 2024

Related U.S. Application Data
(60) Provisional application No. 63/305,211, filed on Jan. 31, 2022.

(51) Int. Cl. A61K 47/69

(52) U.S. Cl. CPC A61K 47/6935

(57) ABSTRACT

The present disclosure provides poly(alkyloxazoline)-lipid conjugates, lipid particles comprising such conjugates, and methods of administering the lipid particles for nucleic acid delivery to cells or subjects.

### CROSS-REFERENCE TO RELATED APPLICATIONS

This application claims priority to U.S. Provisional Application No. 63/305,211, filed Jan. 31, 2022, the disclosure of which is hereby incorporated by reference in its entirety for all purposes.

### BACKGROUND

Lipid nanoparticles (LNP) have become a mainstream pharmaceutical modality with the advent of Onpattro© and mRNA-LNP COVID vaccines. LNP typically have 4 different lipid components, including a cationic lipid, non-cationic lipids such as phospholipids and sterols, and polymer-conjugated lipids. Each has a specific role to play, and they are carefully assembled at specific ratios to yield a highly potent and well-tolerated delivery vehicle.

The cationic lipid becomes positively charged at acidic pH, promoting encapsulation of the negatively charged nucleic acid (e.g., mRNA) payload during particle formation. Following cellular uptake of the LNP, it further drives endosomal fusion and cytoplasmic release of payload. Phospholipids and cholesterol are often referred to as structural lipids, with concentrations chosen to optimize particle size, encapsulation, and stability. The polymer-conjugated lipid controls particle size during formation and prevents particle aggregation by sterically stabilizing the LNP. It is situated at the surface of the particle, with the hydrophilic polymer oriented outwardly, interfacing with the aqueous environment, and the lipid component buried in the particle to anchor it in place.

Throughout the last few decades of development of lipid particles for nucleic acid delivery, the polymer of choice has been poly(ethylene glycol) (PEG) for the polymer-conjugated lipid component. PEG-lipids have been successfully used in several approved nucleic acid-containing LNP, including the COVID-19 vaccines Comirnaty© and SpikeVax. However, the possibility remains that, due to exposure to PEG in other products (e.g., cosmetics), a small subset of the population will have developed antibodies that recognize and bind to PEG, causing hypersensitivity and a loss of drug activity (Judge et al. Mol Ther. 2006, 13(2):328-37). Shortly after the US Food and Drug Administration (FDA) issued Emergency Use Authorization for the above COVID vaccines, cases of anaphylactoid type responses following administration began to be reported (Shimabukuro et al. JAMA. 2021, 325(11):1101-1102). While the cause of these cases has not yet been proven, some have suggested it may be the inclusion of PEG in these products. As such, there is a need for alternate polymers that form effective particles with desirable activity and tolerability profiles. The present disclosure addresses this and other needs.

### BRIEF SUMMARY

Provided herein are poly(alkyloxazoline)-lipid conjugates according to Formula I:

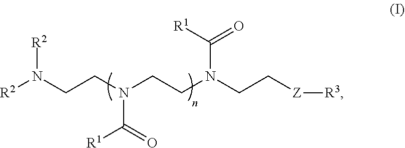

and pharmaceutically acceptable salts thereof, wherein:
subscript n is an integer ranging from 10 to 100;
each R¹ is independently C₁₋₆ alkyl;
each R² is independently selected from the group consisting of hydrogen, C₁₋₆ alkyl, and C₁₋₆ acyl; and
(i) Z is —S(CH₂)₂C(O)—, and
R³ is selected from the group consisting of —NR^{3a}R^{3b}, —NR^{3a}CH₂CH(R^{3b})₂, —CH(R^{3b})₂, and —CH₂CH(R^{3b})₂;
or
(ii) Z is selected from the group consisting of —Z¹—OC(O)—, —Z¹—NHC(O)—, —Z¹—S(O)₂—, and —Z¹—OCH₂—,
Z¹ is selected from the group consisting of a covalent bond, an oligo(ethylene glycol) diradical, and a poly(ethylene glycol) diradical, and
R³ is selected from the group consisting of —NR^{3a}R^{3b} and —CH(R^{3b})₂;
wherein each R^{3a} and R^{3b} is independently selected from the group consisting of hydrogen, C₆₋₂₂ alkyl, C₆₋₂₂ alkenyl, and C₆₋₂₂ alkynyl, wherein one or more non-adjacent CH₂ groups in R^{3a} and R^{3b} are optionally and independently replaced with oxygen,
provided that at least one R^{3a} or R^{3b} is selected from the group consisting of C₆₋₂₂ alkyl, C₆₋₂₂ alkenyl, and C₆₋₂₂ alkynyl, wherein one or more non-adjacent CH₂ groups in R^{3a} or R^{3b} are optionally and independently replaced with oxygen.

In some embodiments, R¹ is ethyl. In some embodiments, R¹ is methyl. In some embodiments, each R¹ is independently selected from the group consisting of methyl and ethyl.

In some embodiments, subscript n is an integer ranging from about 15 to about 55. In some embodiments, the poly(alkyloxazoline) portion of the conjugate has a number average molecular weight ranging from about 2,000 Da to about 5,000 Da.

In some embodiments, the poly(alkyloxazoline)-lipid conjugate has a structure according to Formula IIa:

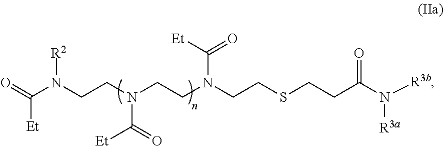

or a pharmaceutically acceptable salt thereof, wherein:
R² is selected from the group consisting of hydrogen and methyl;
R^{3a} is selected from the group consisting of hydrogen, C₁₂₋₁₈ alkyl, and C₁₂₋₁₈ alkenyl; and
R^{3b} is selected from the group consisting of C₁₂₋₁₈ alkyl and C₁₂₋₁₈ alkenyl.

In certain aspects, the present disclosure provides a lipid nanoparticle comprising a poly(alkyloxazoline)-lipid conjugate described herein (e.g., according to Formula I). In some embodiments, the lipid nanoparticle further comprises a cationic lipid, a neutral lipid (e.g., a phospholipid), a sterol (e.g., cholesterol), or a combination thereof. In some embodiments, the lipid nanoparticle further comprises a nucleic acid. In certain embodiments, the nucleic acid comprises an RNA (e.g., an mRNA). In some embodiments, the nucleic acid is fully encapsulated in the lipid nanoparticle. In some embodiments, the lipid nanoparticle has a mean diameter ranging from 40 nm to 150 nm.

In some aspects, the present disclosure provides a lipid nanoparticle comprising:
(a) a nucleic acid;
(b) a cationic lipid comprising from 30 mol % to 80 mol % of the total lipid present in the lipid nanoparticle;
(c) a neutral lipid;
(d) a sterol; and
(e) a poly(alkyloxazoline)-lipid conjugate comprising from 0.1 mol % to 10 mol % of the total lipid present in the lipid nanoparticle.

In some embodiments, the cationic lipid comprises from 40 mol % to 70 mol % of the total lipid present in the lipid nanoparticle. In some embodiments, the cationic lipid comprises from 45 mol % to 65 mol % of the total lipid present in the lipid nanoparticle. In some embodiments, the cationic lipid comprises from 45 mol % to 60 mol % of the total lipid present in the lipid nanoparticle. In some embodiments, the cationic lipid comprises from 50 mol % to 60 mol % of the total lipid present in the lipid nanoparticle.

In some embodiments, the neutral lipid comprises from 3 mol % to 20 mol % of the total lipid present in the lipid nanoparticle. In some embodiments, the neutral lipid comprises from 5 mol % to 15 mol % of the total lipid present in the lipid nanoparticle. In some embodiments, the neutral lipid comprises from 8 mol % to 12 mol % of the total lipid present in the lipid nanoparticle. In certain embodiments, the neutral lipid comprises a phospholipid.

In some embodiments, the sterol comprises from 10 mol % to 60 mol % of the total lipid present in the lipid nanoparticle. In some embodiments, the sterol comprises from 20 mol % to 50 mol % of the total lipid present in the lipid nanoparticle. In some embodiments, the sterol comprises from 30 mol % to 40 mol % of the total lipid present in the lipid nanoparticle. In certain embodiments, the sterol comprises cholesterol.

In some embodiments, the poly(alkyloxazoline)-lipid conjugate comprises from 0.1 mol % to 5 mol % of the total lipid present in the lipid nanoparticle. In some embodiments, the poly(alkyloxazoline)-lipid conjugate comprises from 0.5 mol % to 3 mol % of the total lipid present in the lipid nanoparticle. In certain embodiments, the poly(alkyloxazoline)-lipid conjugate comprises a poly(alkyloxazoline)-lipid conjugate described herein (e.g., according to Formula I).

In particular embodiments, the cationic lipid comprises from 50 mol % to 60 mol % of the total lipid present in the lipid nanoparticle, the neutral lipid comprises from 8 mol % to 12 mol % of the total lipid present in the lipid nanoparticle, the sterol comprises from 30 mol % to 40 mol % of the total lipid present in the lipid nanoparticle, and the poly(alkyloxazoline)-lipid conjugate comprises from 0.5 mol % to 3 mol % of the total lipid present in the lipid nanoparticle.

In some embodiments, the nucleic acid comprises an RNA. In certain embodiments, the RNA comprises an mRNA. In some embodiments, the nucleic acid is fully encapsulated in the lipid nanoparticle. In some embodiments, the lipid nanoparticle has a mean diameter ranging from 40 nm to 150 nm.

In certain aspects, the present disclosure provides a pharmaceutical composition comprising a lipid nanoparticle described herein and a pharmaceutically acceptable carrier. In some embodiments, the pharmaceutical composition is formulated for intravenous, intramuscular, pulmonary, intracerebral, intrathecal, or intranasal administration.

In some aspects, the present disclosure provides a method for introducing a nucleic acid into a cell, the method comprising contacting the cell with a lipid nanoparticle or a pharmaceutical composition described herein. In other aspects, the present disclosure provides a method for delivering a nucleic acid to a subject, the method comprising administering to the subject a lipid nanoparticle or a pharmaceutical composition described herein. In further aspects, the present disclosure provides a method for preventing or treating a disease or disorder in a subject in need thereof, the method comprising administering to the subject a lipid nanoparticle or a pharmaceutical composition described herein. In some embodiments, the disease or disorder is a viral infection, a liver disease or disorder, a lung disease or disorder, a disease or disorder of the CNS, or cancer.

Other objects, features, and advantages of the present disclosure will be apparent to one of skill in the art from the following detailed description.

### DETAILED DESCRIPTION

### I. Introduction

We have designed novel polymer-conjugated lipids comprising a poly(alkyloxazoline) polymer such as poly(2-ethyl 2-oxazoline) (PEOZ). We have conjugated the polymer to various different lipid moieties, arriving at an array of poly(alkyloxazoline)-lipid conjugates. Such lipid conjugates have been formulated into lipid nanoparticles comprising a nucleic acid such as an mRNA payload and characterized as described in the Examples herein.

We have found that the lipid conjugates described herein form effective lipid nanoparticles with favorable physicochemical characteristics such as desirable particle size and low polydispersity as well as high encapsulation efficiency of nucleic acid (e.g., mRNA) payload. In vivo studies demonstrated that the lipid nanoparticle formulations described herein mediate high levels of activity at the target site, exhibiting equivalent or improved activity compared to PEG-lipid conjugates with lower non-specific (off-target) activity. The lipid nanoparticle formulations described herein induce similar or lower cytokine levels compared to PEG-lipid conjugates and thus have a more favorable immunostimulatory profile. The lipid nanoparticle formulations described herein also show improved rate of clearance from the plasma compared to PEG-lipid conjugates and thus have a shorter residence time in the plasma to mitigate antibody responses upon repeat dose administration. Additional in vivo studies demonstrated that the lipid nanoparticle formulations described herein can be successfully incorporated into vaccine platforms to deliver mRNA encoding antigens, inducing antigen-specific immunogenicity at a similar or greater extent than PEG-lipid conjugates. As such, lipid nanoparticles comprising poly(alkyloxazoline)-lipid conjugates provide advantageous activity and tolerability profiles over particles containing PEG-lipid conjugates.

### II. Definitions

As used herein, the singular forms “a,” “an” and “the” include plural referents unless the content clearly dictates otherwise. Thus, for example, reference to “a conjugate” optionally includes a combination of two or more such molecules, and the like.

As used herein, the terms “about” and “approximately,” when used to modify an amount specified in a numeric value or range, indicate that the numeric value as well as reasonable deviations from the value known to the skilled person in the art, for example ±20%, +10%, or +5%, are within the intended meaning of the recited value.

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc.

As used herein, the term “alkenyl,” by itself or as part of another substituent, refers to an alkyl group having at least one carbon-carbon double bond.

As used herein, the term “alkynyl,” by itself or as part of another substituent, refers to an alkyl group having at least one carbon-carbon triple bond.

As used herein, the term “acyl,” by itself or as part of another substituent, refers to a moiety —C(O)R wherein R is an alkyl group.

As used herein, the terms “halo” and “halogen,” by themselves or as part of another substituent, refer to a fluorine, chlorine, bromine, or iodine atom.

As used herein, the term “amino” refers to a moiety —NR₂, wherein each R group is H or alkyl. An amino moiety can be ionized to form the corresponding ammonium cation.

As used herein, the term “sulfonyl” refers to a moiety —SO₂R, wherein the R group is alkyl, haloalkyl, or aryl (e.g., phenyl, toluyl, naphthyl, and the like). An amino moiety can be ionized to form the corresponding ammonium cation. “Alkylsulfonyl” refers to an amino moiety wherein the R group is alkyl.

As used herein, the term “hydroxy” refers to the moiety —OH.

As used herein, the term “salt” refers to a compounds comprising at least one cation (e.g., an organic cation or an inorganic cation) and at least one anion (e.g., an organic anion or an inorganic anion). Acid salts of lipids according to the present disclosure include, but are not limited to, mineral acid salts (e.g., salts formed using hydrochloric acid, hydrobromic acid, phosphoric acid, and the like), organic acid salts (e.g., salts formed using acetic acid, propionic acid, glutamic acid, citric acid, and the like) salts, and quaternary ammonium salts (e.g., salts formed using methyl iodide, ethyl iodide, and the like). Acidic functional groups may be contacted with bases to provide base salts such as alkali and alkaline earth metal salts (e.g., sodium, lithium, potassium, calcium, and magnesium salts), as well as ammonium salts (e.g., ammonium, trimethyl-ammonium, diethylammonium, and tris-(hydroxymethyl)-methyl-ammonium salts).

In some embodiments, the neutral forms of the compounds may be regenerated by contacting the salt with a base or acid and isolating the parent compound in the conventional manner if desired. In some embodiments, the parent form of the compound may differ from various salt forms in certain physical properties, such as solubility in polar solvents, but otherwise the salt forms may be equivalent to the parent form of the compound.

By “pharmaceutically acceptable,” it is meant that the excipient is compatible with the other ingredients of the formulation and is not deleterious to the recipient thereof. It is understood, for example, that pharmaceutically acceptable excipients and salts are non-toxic. Useful pharmaceutical excipients include, but are not limited to, solvents, diluents, pH modifiers, and solubilizers.

The term “nucleic acid” refers to a polymer containing at least two deoxyribonucleotides or ribonucleotides in either single- or double-stranded form and includes DNA and RNA. DNA (e.g., ssDNA or dsDNA) may be in the form of, e.g., antisense molecules, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA (e.g., ssRNA or dsRNA) may be in the form of, e.g., messenger RNA (mRNA), interfering RNA (e.g., small-interfering RNA (siRNA), short hairpin RNA (shRNA), asymmetrical interfering RNA (aiRNA)), microRNA (miRNA), guide RNA (gRNA), self-amplifying RNA, tRNA, rRNA, viral RNA (vRNA), and combinations thereof. In some embodiments, the nucleic acid is a plasmid from which an RNA such as mRNA or an interfering RNA is transcribed. The term encompasses nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the term “lipid particle,” “lipid nanoparticle,” or “LNP” refers to a particle comprising a poly(alkyloxazoline)-lipid conjugate. A lipid particle may comprise additional lipid components such as a cationic lipid and one or more non-cationic lipids (e.g., a phospholipid and/or sterol), and may further comprise a nucleic acid, wherein the nucleic acid may be encapsulated within the particle. In one embodiment, the nucleic acid is at least 50% encapsulated within the particle; in one embodiment, the nucleic acid is at least 75% encapsulated within the particle; in one embodiment, the nucleic acid is at least 90% encapsulated within the particle; and in one embodiment, the nucleic acid is fully encapsulated within the particle. Lipid particles are extremely useful for systemic applications, as they can exhibit extended circulation lifetimes following intravenous injection, they can accumulate at distal sites (e.g., sites physically separated from the administration site), and they can mediate expression of the transfected gene or silencing of the target gene expression at these distal sites. Lipid particles typically have a mean diameter of from about 40 nm to about 150 nm, from about 50 nm to about 150 nm, from about 60 nm to about 130 nm, from about 70 nm to about 110 nm, from about 60 nm to about 100 nm, from about 50 to about 80 nm, from about 60 to about 80 nm, from about 60 to about 90 nm, or from about 70 to about 90 nm, and are substantially non-toxic. In addition, nucleic acids, when present in lipid particles, are resistant in aqueous solution to degradation with a nuclease. Lipid particles and their method of preparation are disclosed in, e.g., U.S. Patent Publication Nos. 2004/0142025 and 2007/0042031.

As used herein, the term “lipid encapsulated” refers to a lipid particle that provides a nucleic acid (e.g., mRNA) with full encapsulation, partial encapsulation, or both. In one embodiment, the nucleic acid is fully encapsulated within the particle.

The term “cationic lipid” refers to a lipid species that carries a net positive charge at a selected pH, such as an acidic pH or physiological pH. Non-limiting examples of cationic lipids are described in detail herein. In some cases, the cationic lipid comprises an ionizable primary, secondary, or tertiary amine (e.g., pH titratable) head group. In one embodiment, the cationic lipid promotes encapsulation of the negatively charged nucleic acid (e.g., mRNA) payload during particle formation. In one embodiment, the cationic lipid drives endosomal fusion and cytoplasmic release of the payload following cellular uptake of the LNP.

The term “non-cationic lipid” includes neutral lipids that exist either in an uncharged or neutral zwitterionic form at a selected pH (e.g., physiological pH), sterols, and anionic lipids that are negatively charged at physiological pH. Non-limiting examples of non-cationic lipids are described in detail herein. Neutral lipids include, e.g., phospholipids such as phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), dipalmitoylphosphatidylcholine (DPPC), dioleoylphosphatidylcholine (DOPC), distearoylphosphatidylcholine (DSPC), and dilinoleoylphosphatidylcholine (DLPC). Sterols include, e.g., cholesterol and derivatives thereof such as cholestanol, cholestanone, cholestenone, coprostanol, cholesteryl-2′-hydroxyethyl ether, and cholesteryl-4′-hydroxybutyl ether. Anionic lipids include, e.g., phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

“Distal site,” as used herein, refers to a physically separated site, which is not limited to an adjacent capillary bed, but includes sites broadly distributed throughout a subject.

“Serum-stable” in relation to lipid particles described herein means that the particle is not significantly degraded after exposure to a serum or nuclease assay that would significantly degrade free DNA or RNA. Suitable assays include, e.g., a standard serum assay, a DNAse assay, or an RNAse assay.

“Systemic delivery,” as used herein, refers to delivery of lipid particles that leads to abroad biodistribution of a nucleic acid such as an mRNA within a subject. Systemic delivery means that a useful, preferably therapeutic, amount of a nucleic acid is exposed to most parts of the body. To obtain broad biodistribution generally requires a blood lifetime such that the nucleic acid is not rapidly degraded or cleared (such as by first pass organs (liver, lung, etc.) or by rapid, nonspecific cell binding) before reaching a target site distal to the site of administration. Systemic delivery of lipid particles can be by any means known in the art including, e.g., intravenous, subcutaneous, and intraperitoneal administration. In one embodiment, lipid particles are delivered intravenously.

“Local delivery,” as used herein, refers to delivery of a nucleic acid such as an mRNA directly to a target site within a subject. For example, a nucleic acid can be locally delivered by direct injection into a disease site such as a tumor or other target site such as a site of inflammation or a target organ such as the liver, heart, pancreas, kidney, and the like.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the subject, individual, or patient is a human.

### III. Poly(Alkyloxazoline)-Lipid Conjugates

Provided herein are poly(alkyloxazoline)-lipid conjugates according to Formula I:

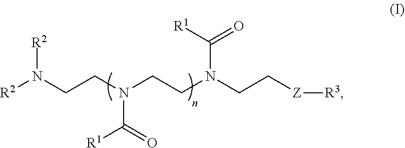

and pharmaceutically acceptable salts thereof, wherein:
subscript n is an integer ranging from 10 to 100;
each R¹ is independently C₁₋₆ alkyl;
each R² is independently selected from the group consisting of hydrogen, C₁₋₆ alkyl, and C₁₋₆ acyl; and
(i) Z is —S(CH₂)₂C(O)—, and
R³ is selected from the group consisting of —NR^{3a}R^{3b}, —NR^{3a}CH₂CH(R^{3b})₂, —CH(R^{3b})₂, and —CH₂CH(R^{3b})₂;
or
(ii) Z is selected from the group consisting of —Z¹—OC(O)—, —Z¹—NHC(O)—, —Z¹—S(O)₂—, and —Z¹—OCH₂—,
Z¹ is selected from the group consisting of a covalent bond, an oligo(ethylene glycol) diradical, and a poly(ethylene glycol) diradical, and
R³ is selected from the group consisting of —NR^{3a}R^{3b} and —CH(R^{3b})₂;
wherein each R^{3a} and R^{3b} is independently selected from the group consisting of hydrogen, C₆₋₂₂ alkyl, C₆₋₂₂ alkenyl, and C₆₋₂₂ alkynyl, wherein one or more non-adjacent CH₂ groups in R^{3a} and R^{3b} are optionally and independently replaced with oxygen,
provided that at least one R^{3a} or R^{3b} is selected from the group consisting of C₆₋₂₂ alkyl, C₆₋₂₂ alkenyl, and C₆₋₂₂ alkynyl, wherein one or more non-adjacent CH₂ groups in R^{3a} or R^{3b} are optionally and independently replaced with oxygen.

In some embodiments, R¹ is ethyl. In some embodiments, R¹ is methyl. In some embodiments, each R¹ is independently selected from the group consisting of methyl and ethyl.

In some embodiments, subscript n is an integer ranging from about 15 to about 55. In some embodiments, the poly(alkyloxazoline) portion of the conjugate has a number average molecular weight ranging from about 2,000 Da to about 5,000 Da. Molecular weights may be determined by any suitable method including, for example, by osmotic pressure, vapor pressure, light scattering, ultracentrifugation, or size exclusion chromatography. Using size exclusion chromatography with an appropriately calibrated column, number average molecular weight Mₙ may be determined according to Equation 1:

$${M}_{n} = \frac{W}{Σ {N}_{i}} = \frac{Σ ( {M}_{i} {N}_{i} )}{Σ {N}_{i}} = \frac{Σ ( {H}_{i} )}{Σ ( {H}_{i} / {M}_{i} )} , \qquad ( 1 )$$

and weight average molecular weight M_{w} may be determined according to Equation 2:

$${M}_{w} = \frac{Σ ( {W}_{i} {M}_{i} )}{W} = \frac{Σ ( {H}_{i} {M}_{i} )}{Σ {H}_{i}} , \qquad ( 2 )$$

wherein W is the total weight of polymers, Wᵢ is the weight of the i^{th} polymer, Mᵢ is the molecular weight of the i^{th} peak in a chromatogram, Nᵢ is the number of molecules with molecular weight Nᵢ, and Hᵢ is the height of the i^{th} peak in the chromatogram.

In some embodiments, the poly(alkyloxazoline)-lipid conjugate has a structure according to Formula IIa:

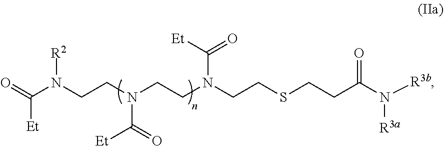

or a pharmaceutically acceptable salt thereof, wherein:
R² is selected from the group consisting of hydrogen and methyl;
R^{3a} is selected from the group consisting of hydrogen, C₁₂₋₁₈ alkyl, and C₁₂₋₁₈ alkenyl; and
R^{3b} is selected from the group consisting of C₁₂₋₁₈ alkyl and C₁₂₋₁₈ alkenyl.

In some embodiments, R^{3a} and R^{3b} are independently selected from the group consisting of C₁₂₋₁₈ alkyl and C₁₂₋₁₈ alkenyl in the poly(alkyloxazoline)-lipid conjugate according to Formula IIa. In some embodiments, R^{3a} and R^{3b} are independently C₁₂₋₁₈ alkyl. In some embodiments, R^{3a} and R^{3b} in Formula IIa are n-dodecyl (lauryl), n-tridecyl, n-tetradecyl (myristyl), n-pentadecyl, n-hexadecyl (cetyl), n-heptadecyl, or n-octadecyl (stearyl). In some embodiments, R^{3a} is H in Formula IIa and R^{3b} is n-dodecyl (lauryl), n-tridecyl, n-tetradecyl (myristyl), n-pentadecyl, n-hexadecyl (cetyl), n-heptadecyl, or n-octadecyl (stearyl).

In some embodiments, the poly(alkyloxazoline)-lipid conjugate has a structure according to Formula IIb:

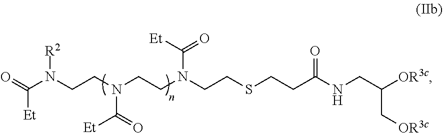

or a pharmaceutically acceptable salt thereof, wherein:
R² is selected from the group consisting of hydrogen and methyl; and
each R³, is independently selected from the group consisting of C₁₂₋₁₈ alkyl and C₁₂₋₁₈ alkenyl. In some embodiments, R^{3a} and R^{3b} in Formula IIb are n-dodecyl (lauryl), n-tridecyl, n-tetradecyl (myristyl), n-pentadecyl, n-hexadecyl (cetyl), n-heptadecyl, or n-octadecyl (stearyl).

Lipid conjugates can be synthesized using the methods described below, together with synthetic methods known in the art of synthetic organic chemistry, or variations thereof. Preferred methods include, but are not limited to, the methods described in the working examples and following schemes. Starting materials and reagents used in preparing the compounds of the present disclosure are either available from commercial suppliers or are prepared by methods known to those skilled in the art following procedures set forth in references such as Fieser and Fieser's Reagents for Organic Synthesis, Vol. 1-28 (Wiley, 2016); March's Advanced Organic Chemistry, 7^{th} Ed. (Wiley, 2013); and Larock's Comprehensive Organic Transformations, 2^{nd} Ed. (Wiley, 1999). The starting materials and the intermediates of the reaction can be isolated and purified if desired using conventional techniques including, but not limited to, filtration, chromatography, crystallization, distillation, and the like. Such materials can be characterized using conventional means, including measuring physical constants and obtaining spectral data.

Conjugates according to Formula IIa and IIb can be formed by reacting an N-hydroxysuccinimidyl ester-terminated polymer according to Formula III (synthesized as described, for example, in WO 2008/106186) with various amines.

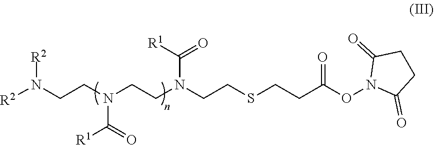

In some embodiments, carbamate-linked conjugates are provided wherein Z is —Z¹—OC(O)—, Z¹ is selected from the group consisting of a covalent bond and a poly(ethylene glycol) diradical, and R³ is —NR^{3a}R^{3b}. Carbamate-linked conjugates may be prepared as shown in the following scheme. A hydroxy-terminated poly(alkyloxazoline) is first reacted with bis(4-nitrophenyl)carbonate to provide a carbonate-terminated poly(alkyloxazoline), which may then be converted to the carbamate-linked product via reaction with a suitable amine. The reactions are typically conducted in the presence of a base such as potassium carbonate, sodium carbonate, sodium acetate, N,N-diisopropylethylamine, lutidines including 2,6-lutidine, triethylamine, tributylamine, pyridine, lithium diisopropylamide, 2,6-di-tert-butylpyridine, 1,8-diazabicycloundec-7-ene (DBU), and the like.

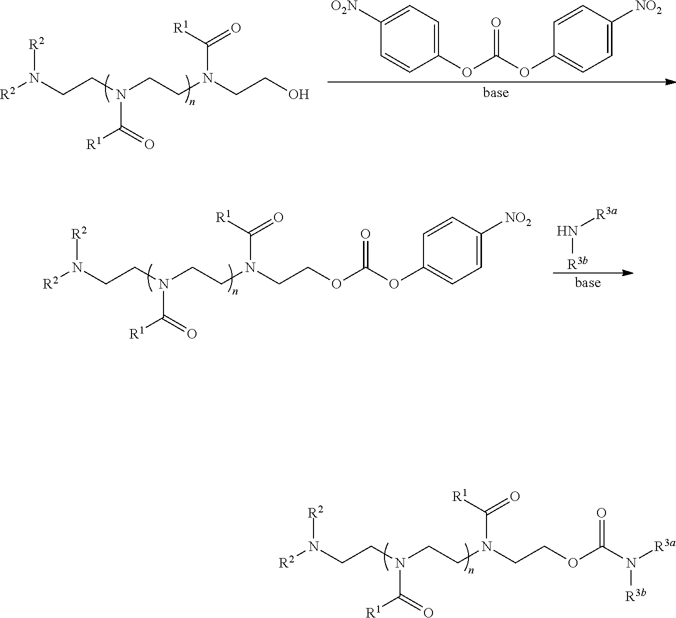

In some embodiments, urea-linked conjugates are provided wherein Z is —Z¹—NHC(O)—, Z¹ is a covalent bond, and R³ is —NR^{3a}R^{3b}. Urea-linked conjugates may be prepared as shown in the following scheme. First a hydroxy-terminated poly(alkyloxazoline) is converted to an amine-terminated poly(alkyloxazoline) by reaction with phthalimide under Mitsunobu conditions and subsequent reduction with hydrazine. The terminal amine is then reacted with triphosgene and an amine HNR^{3a}R^{3b}.

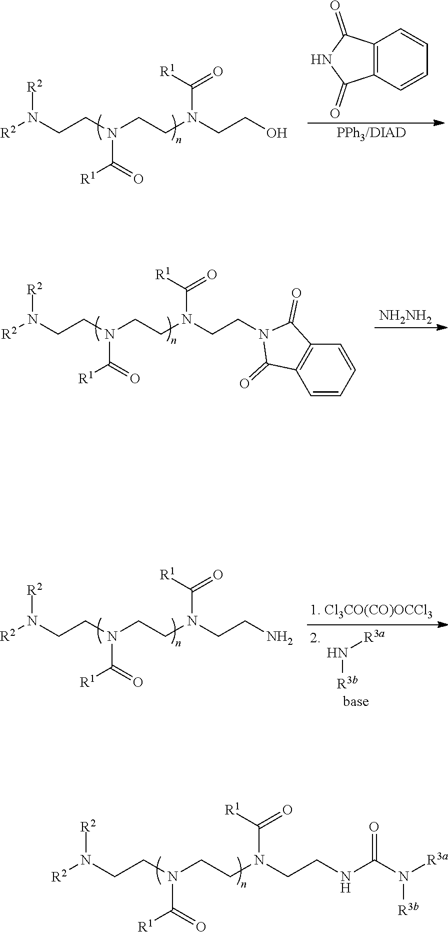

In some embodiments, amide-linked conjugates are provided wherein Z is —Z¹—NHC(O)—, Z¹ is a covalent bond, and R³ is —CH(R^{3b})₂. An amine-terminated poly(alkyloxazoline), prepared as described above, may be acylated with a carboxylic acid to form the amide-linked conjugate as shown in the following scheme. A coupling agent may be used to facilitate amide bond formation. Examples of suitable coupling agents include, but are not limited to, carbodiimides (e.g., N,N′-dicyclohexylcarbodiimide (DCC), 1-ethyl-3-(3-dimethylaminopropyl)-carbodiimide (EDC), and the like), phosphonium salts (e.g., (benzotriazol-1-yloxy)-tripyrrolidinophosphonium hexafluorophosphate (PyBOP); bromotris(dimethylamino)-phosphonium hexafluorophosphate (BroP); and the like); guanidinium/uronium salts (e.g., O-(benzotriazol-1-yl)-N,N,N′,N′-tetramethyluronium hexafluorophosphate (HBTU); 2-(7-aza-1H-benzotriazole-1-yl)-1, 1,3,3-tetramethyluronium hexafluorophosphate (HATU); 1-[(1-(cyano-2-ethoxy-2-oxoethylideneaminooxy) dimethylaminomorpholino)] uronium hexafluorophosphate (COMU); and the like), and propanephosphonic acid anhydride. The coupling agents may be used with bases and/or catalysts including, but not limited to, pyridine and dimethylaminopyridine (DMAP).

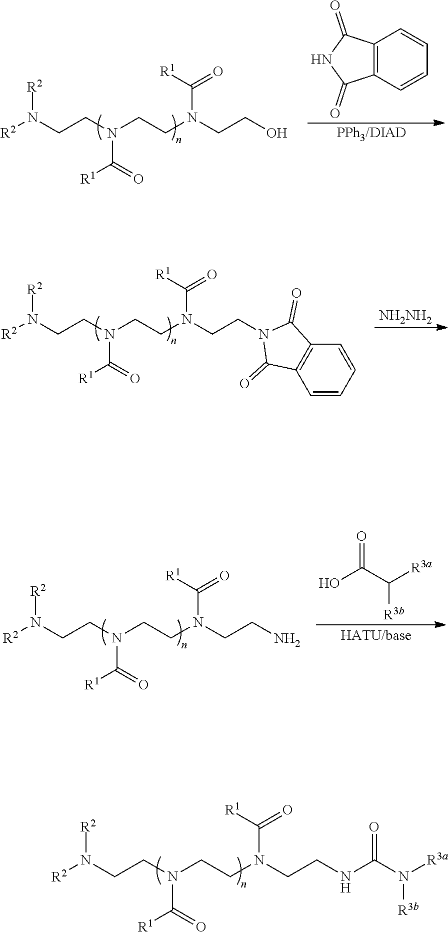

In some embodiments, ester-linked conjugates are provided wherein Z is —Z¹—OC(O)—, Z¹ is a covalent bond, and R³ is —CH(R^{3b})₂. Ester-linked conjugates may be prepared as shown in the following scheme, using a coupling agent for linking a carboxylic acid with a hydroxy-terminated poly(alkyloxazoline).

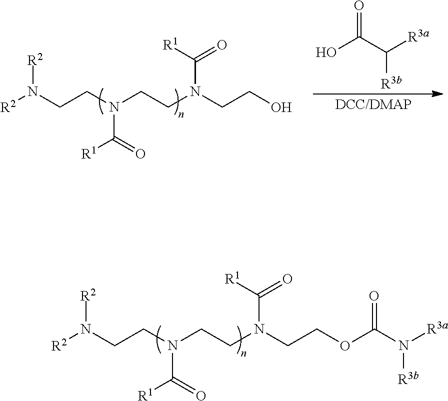

In some embodiments, sulfonamide-linked conjugates are provided wherein Z is —Z¹—S(O)₂—, Z¹ is a covalent bond, and R³ is —NR^{3a}R^{3b}. A hydroxy-terminated poly(alkyloxazoline) can be converted to a sulfonyl chloride-terminated poly(alkyloxazoline) with thionyl chloride and sodium sulfite as shown in the following scheme. The sulfonyl chloride-terminated poly (alkyloxazoline) can then be reacted with an amine in the presence of a base to provide the sulfonamide-linked conjugate.

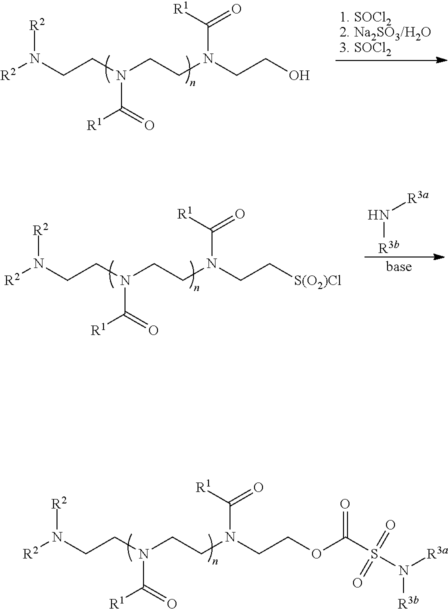

In some embodiments, ether-linked conjugates are provided wherein Z is —Z¹—OCH₂—, Z¹ is a covalent bond, and R³ is —CH(R^{3b})₂. Ether-linked conjugates may be prepared as shown below. A hydroxy-terminated poly(alkyloxazoline) can be converted to a sulfonate-terminated poly(alkyloxazoline), which is reacted with an alcohol to form the ether-linked conjugate. Sulfonates including, but not limited to, mesylate (methanesulfonate), triflate (trifluoro-methanesulfonate), besylate (benzenesulfonate), tosylate (p-toluenesulfonate), and brosylate (4-bromobenzenesulfonate), may be employed.

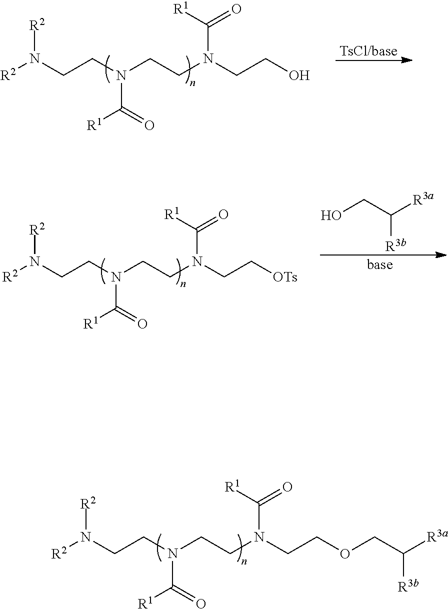

In some embodiments, R^{3a} and each R^{3b} are independently selected from the group consisting of C₁₂₋₁₈ alkyl and C₁₂₋₁₈ alkenyl in the carbamate-linked conjugates, the urea-linked conjugates, the amide-linked conjugates, the ester-linked conjugates, the sulfonamide-linked conjugates, or the ether-linked conjugates. In some embodiments, R^{3a} and each R^{3b} are independently selected C₁₂₋₁₈ alkyl. R^{3a} and R^{3b} may be, for example, n-dodecyl (lauryl), n-tridecyl, n-tetradecyl (myristyl), n-pentadecyl, n-hexadecyl (cetyl), n-heptadecyl, or n-octadecyl (stearyl). In some embodiments, R^{3a} is H and R^{3b} is C₁₂₋₁₈ alkyl. In some embodiments, two R^{3b} are independently selected C₁₂₋₁₈ alkyl.

In some embodiments, poly(alkyloxazoline)-poly(ethylene glycol) lipid conjugates are provided wherein Z is —Z¹—S(CH₂)₂C(O)—, —Z¹—OC(O)—, —Z¹—NHC(O)—, —Z¹—S(O)₂—, or —Z¹—OCH₂—, and Z¹ is an oligo(ethylene glycol) diradical or a poly(ethylene glycol) diradical. The oligo- or poly-(ethylene glycol) portion of the conjugate may contain 2-200 ethylene glycol monomers. In some embodiments, the oligo- or poly-(ethylene glycol) Z¹ contains an average of 3-100 ethylene glycol monomers (e.g., such that the number average molecular weight and/or the weight average molecular weight of Z¹ ranges from 0.025 to 5 kDa, or from 0.5 to 1 kDa). Poly(ethylene glycol) can be functionalized with terminal sulfonate groups, which can then be used for alkylation of hydroxy-terminated poly(alkyloxazoline) to form poly(alkyloxazoline)-poly(ethylene glycol) copolymers as shown below. Various linking strategies as described above can then be employed to prepare the desired lipid conjugates from the copolymers.

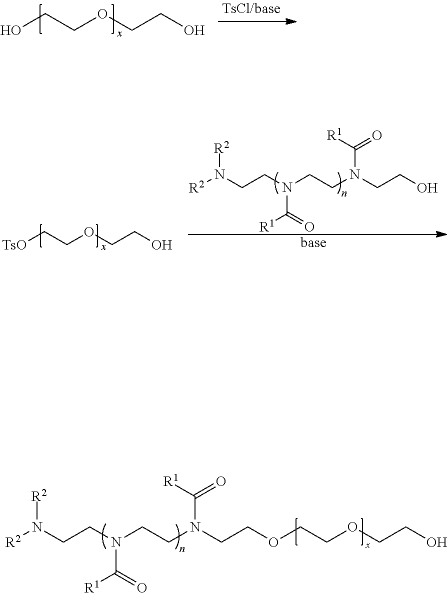

### IV. Lipid Particles

In one aspect, the lipid particle comprises a poly(alkyloxazoline)-lipid conjugate according to Formula I. In some embodiments, the lipid particle further comprises a cationic lipid, a neutral lipid, a sterol, or a combination thereof. In some embodiments, the lipid particle further comprises a nucleic acid. In another aspect, the lipid particle comprises a nucleic acid, a cationic lipid, a neutral lipid, a sterol, and a poly(alkyloxazoline)-lipid conjugate (e.g., according to Formula I). In some embodiments, the nucleic acid is fully encapsulated within the lipid portion of the lipid particle such that the nucleic acid in the lipid particle is resistant in aqueous solution to enzymatic degradation, e.g., by a nuclease or protease. In some embodiments, the lipid particle is substantially non-toxic to mammals such as humans.

In certain embodiments, the present disclosure provides a lipid particle formulation comprising a plurality or population of lipid particles. In some embodiments, the nucleic acid is fully encapsulated within the lipid portion of the lipid particles such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the lipid particles in the plurality or population of lipid particles have the nucleic acid encapsulated therein.

In some embodiments, the lipid particles have a mean diameter ranging from about 40 nm to about 150 nm, from about 50 nm to about 150 nm, from about 60 nm to about 130 nm, from about 70 nm to about 110 nm, from about 60 nm to about 100 nm, from about 50 to about 80 nm, from about 60 to about 80 nm, from about 60 to about 90 nm, or from about 70 to about 90 nm.

In some embodiments, the cationic lipid present in the lipid particle comprises from about 30 mol % to about 80 mol %, from about 40 mol % to about 80 mol %, from about 50 mol % to about 80 mol %, from about 30 mol % to about 70 mol %, from about 40 mol % to about 70 mol %, from about 50 mol % to about 70 mol %, from about 45 mol % to about 80 mol %, from about 45 mol % to about 70 mol %, from about 45 mol % to about 65 mol %, from about 45 mol % to about 60 mol %, from about 45 mol % to about 55 mol %, from about 50 mol % to about 65 mol %, from about 50 mol % to about 60 mol %, or from about 55 mol % to about 65 mol % of the total lipid present in the particle. In some embodiments, the cationic lipid present in the lipid particle comprises about 40 mol %, about 45 mol %, about 50 mol %, about 55 mol %, about 60 mol %, or about 65 mol % of the total lipid present in the particle.

In some embodiments, the neutral lipid (e.g., phospholipid) present in the lipid particle comprises from about 3 mol % to about 20 mol %, from about 5 mol % to about 20 mol %, from about 8 mol % to about 20 mol %, from about 10 mol % to about 20 mol %, from about 3 mol % to about 15 mol %, from about 5 mol % to about 15 mol %, from about 8 mol % to about 15 mol %, from about 10 mol % to about 15 mol %, or from about 8 mol % to about 12 mol % of the total lipid present in the particle. In some embodiments, the neutral lipid (e.g., phospholipid) present in the lipid particle comprises about 5 mol %, about 6 mol %, about 7 mol %, about 8 mol %, about 9 mol %, about 10 mol %, about 11 mol %, about 12 mol %, about 13 mol %, about 14 mol %, or about 15 mol % of the total lipid present in the particle.

In some embodiments, the sterol (e.g., cholesterol) present in the lipid particle comprises from about 10 mol % to about 60 mol %, from about 20 mol % to about 60 mol %, from about 30 mol % to about 60 mol %, from about 40 mol % to about 60 mol %, from about 20 mol % to about 50 mol %, from about 30 mol % to about 50 mol %, from about 35 mol % to about 50 mol %, from about 40 mol % to about 50 mol %, from about 20 mol % to about 45 mol %, from about 25 mol % to about 45 mol %, from about 30 mol % to about 45 mol %, from about 35 mol % to about 45 mol %, from about 20 mol % to about 40 mol %, from about 25 mol % to about 40 mol %, or from about 30 mol % to about 40 mol % of the total lipid present in the particle. In some embodiments, the sterol (e.g., cholesterol) present in the lipid particle comprises about 20 mol %, about 25 mol %, about 30 mol %, about 35 mol %, about 40 mol %, about 45 mol %, or about 50 mol % of the total lipid present in the particle.

In some embodiments, the poly(alkyloxazoline)-lipid conjugate (e.g., according to Formula I) comprises from about 0.1 mol % to about 10 mol %, from about 0.5 mol % to about 10 mol %, from about 1 mol % to about 10 mol %, from about 1.5 mol % to about 10 mol %, from about 2 mol % to about 10 mol %, from about 2.5 mol % to about 10 mol %, from about 3 mol % to about 10 mol %, from about 4 mol % to about 10 mol %, from about 5 mol % to about 10 mol %, from about 0.1 mol % to about 5 mol %, from about 0.3 mol % to about 5 mol %, from about 0.5 mol % to about 5 mol %, from about 1 mol % to about 5 mol %, from about 1.5 mol % to about 5 mol %, from about 2 mol % to about 5 mol %, from about 2.5 mol % to about 5 mol %, from about 3 mol % to about 5 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 2.2 mol % to about 3 mol %, or from about 2.5 mol % to about 3 mol % of the total lipid present in the particle. In some embodiments, the poly(alkyloxazoline)-lipid conjugate (e.g., according to Formula I) present in the lipid particle comprises about 0.1 mol %, about 0.5 mol %, about 1 mol %, about 1.2 mol %, about 1.4 mol %, about 1.5 mol %, about 1.6 mol %, about 1.8 mol %, about 2 mol %, about 2.2 mol %, about 2.5 mol %, or about 3 mol % of the total lipid present in the particle.

In certain embodiments, the lipid particle comprises: a cationic lipid comprising from about 30 mol % to about 80 mol % of the total lipid present in the particle (e.g., from about 40 mol % to about 70 mol %, from about 45 mol % to about 65 mol %, from about 45 mol % to about 60 mol %, or from about 50 mol % to about 60 mol % of the total lipid present in the particle); a neutral lipid such as a phospholipid (e.g., from about 3 mol % to about 20 mol %, from about 5 mol % to about 15 mol %, or from about 8 mol % to about 12 mol % of the total lipid present in the particle); a sterol such as cholesterol (e.g., from about 10 mol % to about 60 mol %, from about 20 mol % to about 50 mol %, or from about 30 mol % to about 40 mol % of the total lipid present in the particle); and a poly(alkyloxazoline)-lipid conjugate (e.g., according to Formula I) comprising from about 0.1 mol % to about 10 mol % of the total lipid present in the particle (e.g., from about 0.1 mol % to about 5 mol % or from about 0.5 mol % to about 3 mol % of the total lipid present in the particle).

In particular embodiments, the lipid particle comprises: a cationic lipid comprising from about 50 mol % to about 60 mol % of the total lipid present in the particle; a neutral lipid (e.g., phospholipid) comprising from about 8 mol % to about 12 mol % of the total lipid present in the particle; a sterol (e.g., cholesterol) comprising from about 30 mol % to about 40 mol % of the total lipid present in the particle; and a poly(alkyloxazoline)-lipid conjugate (e.g., according to Formula I) comprising from about 0.5 mol % to about 3 mol % of the total lipid present in the particle.

In one exemplary embodiment, the lipid particle comprises: a cationic lipid comprising about 55 mol % (e.g., 54.6 mol %) of the total lipid present in the particle; a neutral lipid (e.g., phospholipid) comprising about 11 mol % (e.g., 10.9 mol %) of the total lipid present in the particle; a sterol (e.g., cholesterol) comprising about 33 mol % (e.g., 32.8 mol %) of the total lipid present in the particle; and a poly(alkyloxazoline)-lipid conjugate (e.g., according to Formula I) comprising about 1.6 mol % of the total lipid present in the particle. In certain instances, the lipid particle is formulated into a pharmaceutical composition suitable for intravenous administration.

In another exemplary embodiment, the lipid particle comprises: a cationic lipid comprising about 50.0 mol % of the total lipid present in the particle; a neutral lipid (e.g., phospholipid) comprising about 10 mol % of the total lipid present in the particle; a sterol (e.g., cholesterol) comprising about 38.5 mol % of the total lipid present in the particle; and a poly(alkyloxazoline)-lipid conjugate (e.g., according to Formula I) comprising about 1.5 mol % of the total lipid present in the particle. In certain instances, the lipid particle is formulated into a pharmaceutical composition suitable for intramuscular administration.

In yet another exemplary embodiment, the lipid particle comprises: a cationic lipid comprising about 50.5 mol % of the total lipid present in the particle; a neutral lipid (e.g., phospholipid) comprising about 10 mol % (e.g., 10.1 mol %) of the total lipid present in the particle; a sterol (e.g., cholesterol) comprising about 39 mol % (e.g., 38.9 mol %) of the total lipid present in the particle; and a poly(alkyloxazoline)-lipid conjugate (e.g., according to Formula I) comprising about 0.5 mol % of the total lipid present in the particle.

In a further exemplary embodiment, the lipid particle comprises: a cationic lipid comprising about 49 mol % (e.g., 49.2 mol %) of the total lipid present in the particle; a neutral lipid (e.g., phospholipid) comprising about 10 mol % (e.g., 9.8 mol %) of the total lipid present in the particle; a sterol (e.g., cholesterol) comprising about 38 mol % (e.g., 37.9 mol %) of the total lipid present in the particle; and a poly(alkyloxazoline)-lipid conjugate (e.g., according to Formula I) comprising about 3.0 mol % of the total lipid present in the particle.

Any of a variety of cationic lipids may be used in the lipid particles described herein, either alone or in combination with one or more other cationic lipid species or non-cationic lipid species.

Cationic lipids which are useful in the lipid particles described herein can be any of a number of lipid species which carry a net positive charge at physiological pH. Such lipids include, but are not limited to, N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 1,2-dilinoleyloxy-N,N-dimethylaminopropane (DLinDMA), 1,2-dilinolenyloxy-N,N-dimethylaminopropane (DLenDMA), 3-dimethylamino-2-(cholest-5-en-3-beta-oxybutan-4-oxy)-1-(cis,cis-9,12-octadecadienoxy)propane (CLinDMA), 2-[5′-(cholest-5-en-3.beta.-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-(2-dimethylaminoethyl)-[1,3]-dioxolane (DLin-K-C2-DMA), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), and mixtures thereof.

In certain embodiments, the cationic lipid is (6Z,16Z)-12-((Z)-dec-4-en-1-yl)docosa-6,16-dien-11-yl 5-(dimethylamino)pentanoate having the following structure:

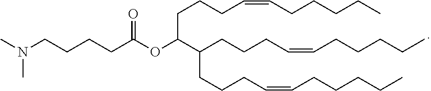 [SMILES: CCCCC/C=C\CCCC(CCC/C=C\CCCCC)C(CCC/C=C\CCCCC)OC(=O)CCCCN(C)C]

In certain other embodiments, the cationic lipid has the following structure:

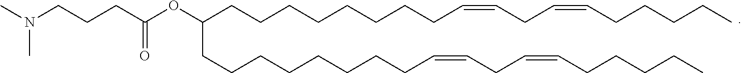 [SMILES: CCCCC/C=C\C/C=C\CCCCCCCCC(CCCCCCCC/C=C\C/C=C\CCCCC)OC(=O)CCCN(C)C]

The non-cationic lipids used in the lipid particles described herein can be any of a variety of neutral uncharged, zwitterionic, or anionic lipids.

Neutral lipids are an exemplary class of non-cationic lipids that include phospholipids such as lecithin, phosphatidylethanolamine, lysolecithin, lysophosphatidylethanolamine (LPE), phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids can be acyl groups derived from fatty acids having C₁₀-C₂₄ carbon chains, e.g., lauroyl, myristoyl, palmitoyl, stearoyl, or oleoyl. In certain embodiments, the neutral lipid comprises DSPC.

Sterols are another exemplary class of non-cationic lipids that include cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the sterol comprises cholesterol.

Any poly(alkyloxazoline)-lipid conjugate according to Formula I may be used in the lipid particles described herein. In one exemplary embodiment, the poly(alkyloxazoline) portion of the conjugate comprises a poly(2-ethyl 2-oxazoline) (PEOZ) polymer. In some embodiments, the poly(alkyloxazoline) portion of the conjugate has an average molecular weight ranging from about 2,000 Da to about 5,000 Da. In particular embodiments, the poly(alkyloxazoline) portion of the conjugate has an average molecular weight of about 5,000 Da. In some embodiments, the lipid portion of the conjugate comprises one or two independently selected C₁₂₋₁₈ alkyl chains. In particular embodiments, the lipid portion of the conjugate comprises one or two independently selected C₁₂, C₁₄, C₁₆, or C₁₈ alkyl chains. Additional poly(alkyloxazoline)-lipid conjugates suitable for use in the lipid particles described herein include the poly(alkyloxazoline)-dialkyloxypropyl conjugates disclosed in U.S. Patent Publication No. 2011/0313017 and the poly(alkyloxazoline)-phospholipid conjugates disclosed in International Publication No. WO 2010/006282.

### V. Nucleic Acids

In certain embodiments, the lipid particles described herein are associated with a nucleic acid, resulting in a nucleic acid-lipid particle. In some embodiments, the nucleic acid is fully encapsulated in the lipid particle. As used herein, the term “nucleic acid” includes any oligonucleotide or polynucleotide, with fragments containing up to 60 nucleotides generally termed oligonucleotides, and longer fragments termed polynucleotides. Nucleic acid may be administered alone in the lipid particles described herein, or in combination (e.g., co-administered) with lipid particles comprising peptides, polypeptides, or small molecules such as conventional drugs.

As used herein, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted polynucleotides and oligonucleotides are often preferred over native forms because of properties such as, for example, enhanced cellular uptake, reduced immunogenicity, and increased stability in the presence of nucleases.

Oligonucleotides are generally classified as deoxyribooligonucleotides or ribooligonucleotides. A deoxyribooligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose.

The nucleic acid that is present in the lipid particles described herein includes any form of nucleic acid that is known. The nucleic acids used herein can be single-stranded DNA or RNA (e.g., ssDNA or ssRNA), or double-stranded DNA or RNA (e.g., dsDNA or dsRNA), or DNA-RNA hybrids. Single-stranded nucleic acids include, e.g., mRNA, guide RNA (gRNA), antisense oligonucleotides, ribozymes, mature miRNA, and triplex-forming oligonucleotides. Examples of double-stranded DNA include, e.g., structural genes, genes including control and termination regions, and self-replicating systems such as viral or plasmid DNA. Examples of double-stranded RNA include, e.g., siRNA and other RNAi agents such as aiRNA and pre-miRNA.

Nucleic acids may be of various lengths, generally dependent upon the particular form of nucleic acid. For example, in particular embodiments, mRNA, plasmids, or genes may be from about 1,000 to about 100,000 nucleotides in length. In particular embodiments, oligonucleotides may range from about 10 to about 100 nucleotides in length. In various related embodiments, oligonucleotides, both single-stranded, double-stranded, and triple-stranded, may range in length from about 10 to about 60 nucleotides, from about 15 to about 60 nucleotides, from about 20 to about 50 nucleotides, from about 15 to about 30 nucleotides, or from about 20 to about 30 nucleotides in length.

In particular embodiments, an oligonucleotide (or a strand thereof) specifically hybridizes to or is complementary to a target polynucleotide sequence. The terms “specifically hybridizable” and “complementary” as used herein indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. In some embodiments, an oligonucleotide is specifically hybridizable when binding of the oligonucleotide to the target sequence interferes with the normal function of the target sequence to cause a loss of utility or expression therefrom, and there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under conditions in which the assays are conducted. Thus, the oligonucleotide may include 1, 2, 3, or more base substitutions as compared to the region of a gene or mRNA sequence that it is targeting or to which it specifically hybridizes.

A. mRNA

Certain embodiments provide compositions comprising the lipid particles described herein and methods of use thereof for expressing one or more mRNA molecules (e.g., a cocktail of mRNA molecules) in a cell (e.g., a cell within a human body). The mRNA molecules encode one or more polypeptides that is/are expressed within the cell. Lipid particle formulations comprising mRNA molecules described herein are useful for a variety of applications including protein replacement therapy, vaccines, cancer immunotherapy, and gene editing. In some embodiments, the lipid particles described herein are used for treating a disease, wherein expression of the polypeptides encoded by the mRNA molecules within a diseased organism (e.g., a mammal, such as a human) ameliorates one or more symptoms of the disease. The compositions and methods described herein are particularly useful for treating human diseases caused by the absence, or reduced levels, of a functional polypeptide within the human body. In other embodiments, the lipid particles described herein are used as a vaccine for preventing a disease, wherein expression of the polypeptides encoded by the mRNA molecules within an organism (e.g., a mammal, such as a human) elicits immunity against the disease. The compositions and methods described herein are particularly useful for preventing an infectious disease caused by a pathogen such as a virus (e.g., a coronavirus such as SARS-CoV-2) by expressing antigenic polypeptides (e.g., from mRNA molecules encoding viral proteins such as S (spike), E (envelope), M (membrane), or N (nucleocapsid) proteins or antigenic fragments thereof) to produce an immune response within an organism (e.g., a mammal, such as a human) by stimulating the adaptive immune system to create antibodies that target the pathogen. In yet other embodiments, the lipid particles described herein are used as a vaccine for treating a disease, wherein expression of the polypeptides encoded by the mRNA molecules within an organism (e.g., a mammal, such as a human) elicits an immune response against diseased cells. The compositions and methods described herein are particularly useful for treating cancer by expressing antigenic polypeptides (e.g., from mRNA molecules encoding tumor-specific antigens or antigenic fragments thereof) to stimulate an adaptive immune response to create antibodies that target and destroy cancer cells.

In some embodiments, the mRNA molecules are fully encapsulated in lipid particle. With respect to formulations comprising an mRNA cocktail, the different types of mRNA species present in the cocktail (e.g., mRNA having different sequences) may be co-encapsulated in the same particle, or each type of mRNA species present in the cocktail may be encapsulated in a separate particle. The mRNA cocktail may be formulated in the particles described herein using a mixture of two or more individual mRNAs (each having a unique sequence) at identical, similar, or different concentrations or molar ratios. In one embodiment, a cocktail of mRNAs (corresponding to a plurality of mRNAs with different sequences) is formulated using identical, similar, or different concentrations or molar ratios of each mRNA species, and the different types of mRNAs are co-encapsulated in the same particle. In another embodiment, each type of mRNA species present in the cocktail is encapsulated in different particles at identical, similar, or different mRNA concentrations or molar ratios, and the particles thus formed (each containing a different mRNA payload) are administered separately (e.g., at different times in accordance with a prophylactic or therapeutic regimen), or are combined and administered together as a single unit dose (e.g., with a pharmaceutically acceptable carrier). In particular embodiments, the lipid particles are serum-stable, are resistant to nuclease degradation, and are substantially non-toxic to mammals such as humans.

1. Modifications to mRNA

The mRNA molecules present in the lipid particles can include one, two, or more than two nucleoside modifications. In some embodiments, the modified mRNA exhibits reduced degradation in a cell into which the mRNA is introduced, relative to a corresponding unmodified mRNA.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihy drouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In certain embodiments, the modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine, or 5′-O-(1-thiophosphate)-pseudouridine. The α-thio substituted phosphate moiety is provided to confer stability to RNA polymers through the unnatural phosphorothioate backbone linkages. Phosphorothioate RNA have increased nuclease resistance and subsequently a longer half-life in a cellular environment. Phosphorothioate-linked nucleic acids are expected to also reduce the innate immune response through weaker binding/activation of cellular innate immune molecules.

In certain embodiments, it is desirable to intracellularly degrade a modified nucleic acid introduced into the cell, for example, if precise timing of protein production is desired. Thus, the present disclosure provides a modified nucleic acid containing a degradation domain, which is capable of being acted on in a directed manner within a cell.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

2. Optional mRNA Components

In further embodiments, the mRNA molecules present in the lipid particles may include other optional components. These optional components include, but are not limited to, untranslated regions, Kozak sequences, intronic nucleotide sequences, internal ribosome entry site (IRES), caps, and poly-A tails. For example, a 5′ untranslated region (UTR) and/or a 3′ UTR may be included, wherein either or both may independently contain one or more different nucleoside modifications. In such embodiments, nucleoside modifications may also be present in the translatable region. Also provided are mRNA molecules containing a Kozak sequence. Additionally, provided herein are mRNA molecules containing one or more intronic nucleotide sequences capable of being excised from the mRNA sequence.

### A. Untranslated Regions (UTRs)

Untranslated regions (UTRs) of a gene are transcribed but not translated. The 5′ UTR starts at the transcription start site and continues to the start codon but does not include the start codon; whereas, the 3′ UTR starts immediately following the stop codon and continues until the transcriptional termination signal. There is a growing body of evidence about the regulatory roles played by the UTRs in terms of stability of the mRNA molecule and translation. The regulatory features of a UTR can be incorporated into the mRNA used in the lipid particles described herein to increase the stability of the molecule. The specific features can also be incorporated to ensure controlled downregulation of the transcript in case they are misdirected to undesired tissue or organ sites.

### b. 5′ Capping

The 5′ cap structure of an mRNA is involved in nuclear export, increasing mRNA stability, and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped, generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or antiterminal transcribed nucleotides of the 5′ end of the mRNA may optionally also be 2′-O-methylated. 5′-decapping through hydrolysis and cleavage of the guanylate cap structure may target an mRNA molecule for degradation.

c. IRES Sequences

mRNA containing an internal ribosome entry site (IRES) are also useful in the lipid particles described herein. An TRES may act as the sole ribosome binding site, or may serve as one of multiple ribosome binding sites of an mRNA. An mRNA containing more than one functional ribosome binding site may encode several peptides or polypeptides that are translated independently by the ribosomes (“multicistronic mRNA”). When mRNA are provided with an TRES, further optionally provided is a second translatable region. Examples of RES sequences include, without limitation, those from picomaviruses (e.g., FMDV), pest viruses (e.g., CFFV), polio viruses (PV), encephalomyocarditis viruses (ECMV), foot-and-mouth disease viruses (FMDV), hepatitis C viruses (HCV), classical swine fever viruses (CSFV), murine leukemia viruses (MLV), simian immune deficiency viruses (SlV), and cricket paralysis viruses (CrPV).

d. Poly-A Tails

During RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between 100 and 250 residues long.

Generally, the length of a poly-A tail is greater than 30 nucleotides in length. In some embodiments, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1100, 1200, 1300, 1400, 1500, 1600, 1700, 1800, 1900, 2,000, 2,500, and 3,000 nucleotides). In some embodiments, the poly-A tail may be 10%, 20%, 30%, 40%, 50%, 60%, 70%, 80%, 90%, or 100% greater in length than the mRNA. In other embodiments, the poly-A tail may be 10%, 20%, 30%, 40%, 50%, 60%, 70%, 80%, 90%, or more of the total length of the mRNA.

3. Generating mRNA Molecules

Methods for isolating RNA, synthesizing RNA, hybridizing nucleic acids, making and screening cDNA libraries, and performing PCR are well known in the art (see, e.g., Gubler and Hoffman, Gene, 25:263-269 (1983); Sambrook et al., Molecular Cloning, A Laboratory Manual (2nd ed. 1989)) as are PCR methods (see, e.g., U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in the present disclosure include Kriegler, Gene Transfer and Expression: A Laboratory Manual (1990); and Current Protocols in Molecular Biology (Ausubel et al., eds., 1994).

4. Applications of mRNA Therapy

The mRNA component of the lipid particles described herein can be used to express a polypeptide of interest. Certain diseases in humans are caused by the absence or impairment of a functional protein in a cell type where the protein is normally present and active. The functional protein can be completely or partially absent due, e.g., to transcriptional inactivity of the encoding gene or due to the presence of a mutation in the encoding gene that renders the protein completely or partially non-functional. Examples of human diseases that are caused by complete or partial inactivation of a protein include methylmalonic academia (caused by defective methylmalonyl-CoA mutase), glycogen storage disease type 1A (caused by a defective catalytic subunit of glucose-6-phosphatase), glycogen storage disease type 1B (caused by a lack of glucose-6-phosphate translocase), fragile X syndrome (caused by a deficiency of FMR1 protein), urea cycle disorder (caused by mutations in the ornithine transcarbamoylase (OTC) gene), Crigler-Najjar syndrome type 1 (caused by a genetic mutation leading to the lack of bilirubin uridine diphosphate glucuronosyltransferase (bilirubin-UGT)), alpha-1 antitrypsin deficiency (caused by mutations in the SERPINA1 gene), thrombotic thrombocytopenic purpura (caused by mutations in a disintegrin and metalloproteinase with a thrombospondin type 1 motif, member 13 (ADAMTS13) gene), acute intermittent porphyria (caused by a genetic mutation of the PBGD locus), Factor IX deficiency hemophilia B (caused by a deficiency of factor IX (FIX) protein), X-linked severe combined immunodeficiency (X-SCID) (caused by one or more mutations in the gene encoding the common gamma chain protein that is a component of the receptors for several interleukins that are involved in the development and maturation of B and T cells within the immune system), and X-linked adrenoleukodystrophy (X-ALD) (caused by one or more mutations in a peroxisomal membrane transporter protein gene called ABCD1).

In some embodiments, the mRNA component of the lipid particles described herein expresses an infectious disease antigen such as a viral, bacterial, fungal, protozoal, and/or helminthic infectious disease antigen. Such vaccines comprising lipid particles with antigen-encoding mRNA are particularly useful for preventing or treating the infectious disease. In certain embodiments, the infectious disease antigen is a viral infectious disease antigen from a coronavirus (e.g., SARS-CoV-1, SARS-CoV-2, MERS-CoV), influenza virus (e.g., influenza A, B, and C viruses), filovirus (e.g., Ebola virus, Marburg virus), arenavirus (e.g., Lassa virus, Junin virus, Machupo virus, Guanarito virus, Sabia virus), Zika virus, rabies virus, rhinovirus, simian immunodeficiency virus (SIV), human immunodeficiency virus (HIV), hepatitis viruses (e.g., hepatitis C virus), herpes simplex virus, human papilloma virus (HPV), or Epstein-Barr virus. In particular embodiments, the infectious disease antigen is a SARS-CoV-2 protein selected from the group consisting of S (spike) protein, E (envelope) protein, M (membrane) protein, N (nucleocapsid) protein, and an antigenic fragment thereof. In certain instances, the development of antigen-specific immunity from an mRNA vaccine requires the transfection of antigen-presenting cells, such as dendritic cells. Administration is typically accomplished by intradermal, intramuscular or subcutaneous injection, as dendritic cells densely populate skin tissue and skeletal muscle.

In some embodiments, the mRNA component of the lipid particles described herein expresses a tumor-associated antigen. In certain instances, following the administration of an mRNA cancer vaccine to dendritic cells, cytotoxic T cells can target and destroy tumors. In other embodiments, the mRNA component of a lipid particle described herein expresses a chimeric antigen receptor (CAR) for CAR T cell therapy. Typically, a subject's T cells are isolated and transfected ex vivo with mRNA encoding CARs, which are protein fragments that are displayed on the T cell surface and bind to specific tumor epitopes. Following the re-introduction of the modified T cells into a subject, the CARs target and kill tumor cells.

In some embodiments, the mRNA component of the lipid particles described herein expresses a gene editing nuclease. Examples of gene editing nucleases include CRISPR/Cas nucleases (e.g., Cas9, Cpf1), zinc finger nucleases (ZFNs), transcription-activator like effector nucleases (TALEN), and meganucleases. CRISPR-mediated gene editing requires a Cas nuclease responsible for DNA cleavage and a short guide RNA (gRNA) that directs the Cas nuclease to cleave the DNA at a precise location. In some embodiments, the gRNA targets the Cas nuclease to a gene in a viral genome. As a non-limiting example, the viral genome is a SARS-CoV-2 genome and the gene is selected from the group consisting of orflab, S gene, ORF3a, E gene, M gene, ORF6, ORF7a, ORF8, N gene, and ORF10. In certain instances, an mRNA encoding a Cas nuclease such as Cas9 and a gRNA are encapsulated in the same lipid particle. In other instances, the mRNA encoding the Cas nuclease and the gRNA are encapsulated in separate lipid particles.

B. siRNA

The siRNA component of the lipid particles described herein is capable of silencing the expression of a target gene of interest. Each strand of the siRNA duplex is typically about 15 to about 60 nucleotides in length, preferably about 15 to about 30 nucleotides in length. In certain embodiments, the siRNA comprises at least one modified nucleotide. The modified siRNA is generally less immunostimulatory than a corresponding unmodified siRNA sequence and retains RNAi activity against the target gene of interest. In some embodiments, the modified siRNA contains at least one 2′OMe purine or pyrimidine nucleotide such as a 2′OMe-guanosine, 2′OMe-uridine, 2′OMe-adenosine, and/or 2′OMe-cytosine nucleotide. The modified nucleotides can be present in one strand (i.e., sense or antisense) or both strands of the siRNA. The siRNA sequences may have overhangs (e.g., 3′ or 5′ overhangs as described in Elbashir et al., Genes Dev., 15:188 (2001) or Nykänen et al., Cell, 107:309 (2001)), or may lack overhangs (i.e., have blunt ends).

Suitable siRNA sequences can be identified using any means known in the art. Typically, the methods described in Elbashir et al., Nature, 411:494-498 (2001) and Elbashir et al., EMBO J., 20:6877-6888 (2001) are combined with rational design rules set forth in Reynolds et al., Nature Biotech., 22(3):326-330 (2004).

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. siRNA can be chemically synthesized. The oligonucleotides that comprise the siRNA molecules can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997).

Examples of modified nucleotides include, but are not limited to, ribonucleotides having a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl group. Modified nucleotides having a Northern conformation such as those described in, e.g., Saenger, Principles of Nucleic Acid Structure, Springer-Verlag Ed. (1984), are also suitable for use in siRNA molecules. Such modified nucleotides include, without limitation, locked nucleic acid (LNA) nucleotides (e.g., 2′-0, 4′—C-methylene-(D-ribofuranosyl) nucleotides), 2′-O-(2-methoxyethyl) (MOE) nucleotides, 2′-methyl-thio-ethyl nucleotides, 2′-deoxy-2′-fluoro (2′F) nucleotides, 2′-deoxy-2′-chloro (2′Cl) nucleotides, and 2′-azido nucleotides. In certain instances, the siRNA molecules described herein include one or more G-clamp nucleotides. A G-clamp nucleotide refers to a modified cytosine analog wherein the modifications confer the ability to hydrogen bond both Watson-Crick and Hoogsteen faces of a complementary guanine nucleotide within a duplex (see, e.g., Lin et al., J. Am. Chem. Soc., 120:8531-8532 (1998)). In addition, nucleotides having a nucleotide base analog such as, for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (see, e.g., Loakes, Nucl. Acids Res., 29:2437-2447 (2001)) can be incorporated into siRNA molecules.

In certain embodiments, siRNA molecules may further comprise one or more chemical modifications such as terminal cap moieties, phosphate backbone modifications, and the like. Examples of terminal cap moieties include, without limitation, inverted deoxy abasic residues, glyceryl modifications, 4′,5′-methylene nucleotides, 1-(D-D-erythrofuranosyl) nucleotides, 4′-thio nucleotides, carbocyclic nucleotides, 1,5-anhydrohexitol nucleotides, L-nucleotides, α-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al., Tetrahedron 49:1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al., Nucleic Acid Analogues: Synthesis and Properties, in Modern Synthetic Methods, VCH, 331-417 (1995); Mesmaeker et al., Novel Backbone Replacements for Oligonucleotides, in Carbohydrate Modifications in Antisense Research, ACS, 24-39 (1994)). Such chemical modifications can occur at the 5′-end and/or 3′-end of the sense strand, antisense strand, or both strands of the siRNA.

In some embodiments, the sense and/or antisense strand of the siRNA molecule can further comprise a 3′-terminal overhang having about 1 to about 4 (e.g., 1, 2, 3, or 4) 2′-deoxy ribonucleotides and/or any combination of modified and unmodified nucleotides. Additional examples of modified nucleotides and types of chemical modifications that can be introduced into siRNA molecules are described, e.g., in UK Patent No. GB 2,397,818 B and U.S. Patent Publication Nos. 20040192626, 20050282188, and 20070135372.

The siRNA molecules described herein can optionally comprise one or more non-nucleotides in one or both strands of the siRNA. As used herein, the term “non-nucleotide” refers to any group or compound that can be incorporated into a nucleic acid chain in the place of one or more nucleotide units, including sugar and/or phosphate substitutions, and allows the remaining bases to exhibit their activity. The group or compound is abasic in that it does not contain a commonly recognized nucleotide base such as adenosine, guanine, cytosine, uracil, or thymine and therefore lacks a base at the 1′-position.

In other embodiments, chemical modification of the siRNA comprises attaching a conjugate to the siRNA molecule. The conjugate can be attached at the 5′ and/or 3′-end of the sense and/or antisense strand of the siRNA via a covalent attachment such as, e.g., a biodegradable linker. The conjugate can also be attached to the siRNA, e.g., through a carbamate group or other linking group (see, e.g., U.S. Patent Publication Nos. 20050074771, 20050043219, and 20050158727). In certain instances, the conjugate is a molecule that facilitates the delivery of the siRNA into a cell. Examples of conjugate molecules suitable for attachment to siRNA include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Yet other examples include the 2′-O-alkyl amine, 2′-O-alkoxyalkyl amine, polyamine, C5-cationic modified pyrimidine, cationic peptide, guanidinium group, amidinium group, cationic amino acid conjugate molecules described in U.S. Patent Publication No. 20050153337. Additional examples include the hydrophobic group, membrane active compound, cell penetrating compound, cell targeting signal, interaction modifier, and steric stabilizer conjugate molecules described in U.S. Patent Publication No. 20040167090. Further examples include the conjugate molecules described in U.S. Patent Publication No. 20050239739. The type of conjugate used and the extent of conjugation to the siRNA molecule can be evaluated for improved pharmacokinetic profiles, bioavailability, and/or stability of the siRNA while retaining RNAi activity. As such, one skilled in the art can screen siRNA molecules having various conjugates attached thereto to identify ones having improved properties and full RNAi activity using any of a variety of well-known in vitro cell culture or in vivo animal models.

The siRNA component of the lipid particles described herein can be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation (e.g., cancer), angiogenic genes, immunomodulator genes such as those associated with inflammatory and autoimmune responses, ligand receptor genes, and genes associated with neurodegenerative disorders.

Genes associated with viral infection and survival include those expressed by a virus in order to bind, enter, and replicate in a cell. Viral sequences of particular interest include sequences from a coronavirus (e.g., SARS-CoV-1, SARS-CoV-2, MERS-CoV), influenza virus (e.g., influenza A, B, and C viruses), filovirus (e.g., Ebola virus, Marburg virus), arenavirus (e.g., Lassa virus, Junin virus, Machupo virus, Guanarito virus, Sabia virus), Zika virus, rabies virus, rhinovirus, simian immunodeficiency virus (SIV), human immunodeficiency virus (HIV), hepatitis viruses (e.g., hepatitis C virus), herpes simplex virus, human papilloma virus (HPV), or Epstein-Barr virus. Exemplary coronavirus (e.g., SARS-CoV-2) nucleic acid sequences that can be silenced include, but are not limited to, nucleic acid sequences encoding S (spike) protein, E (envelope) protein, M (membrane) protein, and N (nucleocapsid) protein. Exemplary filovirus nucleic acid sequences that can be silenced include, but are not limited to, nucleic acid sequences encoding structural proteins (e.g., VP30, VP35, nucleoprotein (NP), polymerase protein (L-pol)) and membrane-associated proteins (e.g., VP40, glycoprotein (GP), VP24). Exemplary influenza virus nucleic acid sequences that can be silenced include, but are not limited to, nucleic acid sequences encoding nucleoprotein (NP), matrix proteins (M1 and M2), nonstructural proteins (NS1 and NS2), RNA polymerase (PA, PB1, PB2), neuraminidase (NA), and haemagglutinin (HA). Exemplary hepatitis virus nucleic acid sequences that can be silenced include, but are not limited to, nucleic acid sequences involved in transcription and translation (e.g., En1, En2, X, P) and nucleic acid sequences encoding structural proteins (e.g., core proteins including C and C-related proteins, capsid and envelope proteins including S, M, and/or L proteins, or fragments thereof).

Genes associated with metabolic diseases and disorders (e.g., disorders in which the liver is the target and liver diseases and disorders) include, for example, genes expressed in dyslipidemia (e.g., liver X receptors such as LXRα and LXRβ, farnesoid X receptors (FXR), sterol-regulatory element binding protein (SREBP), site-1 protease (SiP), 3-hydroxy-3-methylglutaryl coenzyme-A reductase (HMG coenzyme-A reductase), apolipoprotein B (ApoB), apolipoprotein CIII (ApoC3), and apolipoprotein E (ApoE); and diabetes (e.g., glucose 6-phosphatase). One of skill in the art will appreciate that genes associated with metabolic diseases and disorders (e.g., diseases and disorders in which the liver is a target and liver diseases and disorders) include genes that are expressed in the liver itself as well as and genes expressed in other organs and tissues. Silencing of sequences that encode genes associated with metabolic diseases and disorders can conveniently be used in combination with the administration of conventional agents used to treat the disease or disorder.

Examples of gene sequences associated with tumorigenesis and cell transformation (e.g., cancer or other neoplasia) include mitotic kinesins such as Eg5 (KSP, KIF11); serine/threonine kinases such as polo-like kinase 1 (PLK-1); tyrosine kinases such as WEEl; inhibitors of apoptosis such as XIAP; COP9 signalosome subunits such as CSN1, CSN2, CSN3, CSN4, CSN5; CSN6, CSN7A, CSN7B, and CSN8; ubiquitin ligases such as COP1 (RFWD2); and histone deacetylases such as HDAC1, HDAC2, HDAC3, HDAC4, HDAC5, HDAC6, HDAC7, HDAC8, HDAC9, etc.

Additional examples of gene sequences associated with tumorigenesis and cell transformation include translocation sequences such as MLL fusion genes, BCR-ABL, TEL-AML1, EWS-FLI1, TLS-FUS, PAX3-FKHR, BCL-2, AML1-ETO, and AML1-MTG8; overexpressed sequences such as multidrug resistance genes, cyclins, beta-catenin, telomerase genes, c-MYC, N-MYC, BCL-2, growth factor receptors (e.g., EGFR/ErbB1, ErbB2/HER-2, ErbB3, and ErbB4); and mutated sequences such as RAS.

Silencing of sequences that encode DNA repair enzymes find use in combination with the administration of chemotherapeutic agents. Genes encoding proteins associated with tumor migration are also target sequences of interest, for example, integrins, selectins, and metalloproteinases. The foregoing examples are not exclusive. Those of skill in the art will understand that any whole or partial gene sequence that facilitates or promotes tumorigenesis or cell transformation, tumor growth, or tumor migration can be included as a template sequence.

Angiogenic genes are able to promote the formation of new vessels. Of particular interest is vascular endothelial growth factor (VEGF) or VEGFR. Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin, angiostatin, and VEGFR2.

Immunomodulator genes are genes that modulate one or more immune responses. Examples of immunomodulator genes include, without limitation, cytokines such as growth factors (e.g., TGF-α, TGF-β, EGF, FGF, IGF, NGF, PDGF, CGF, GM-CSF, SCF, etc.), interleukins (e.g., IL-2, IL-4, IL-12, IL-15, IL-18, IL-20, etc.), interferons (e.g., IFN-α, IFN-β, IFN-γ, etc.) and TNF. Fas and Fas ligand genes are also immunomodulator target sequences of interest. Genes encoding secondary signaling molecules in hematopoietic and lymphoid cells are also included, for example, Tec family kinases such as Bruton's tyrosine kinase (Btk).

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g., inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, glucagon, G-protein coupled receptor ligands, etc. Templates coding for an expansion of trinucleotide repeats (e.g., CAG repeats) find use in silencing pathogenic sequences in neurodegenerative disorders caused by the expansion of trinucleotide repeats, such as spinobulbular muscular atrophy and Huntington's Disease.

Certain other target genes, which may be targeted by a nucleic acid (e.g., siRNA) to downregulate or silence the expression of the gene, include but are not limited to, Actin, Alpha 2, Smooth Muscle, Aorta (ACTA2), Alcohol dehydrogenase 1A (ADH1A), Alcohol dehydrogenase 4 (ADH4), Alcohol dehydrogenase 6 (ADH6), Afamin (AFM), Angiotensinogen (AGT), Serine-pyruvate aminotransferase (AGXT), Alpha-2-HS-glycoprotein (AHSG), Aldo-keto reductase family 1 member C4 (AKR1C4), Serum albumin (ALB), alpha-i-microglobulin/bikunin precursor (AMBP), Angiopoietin-related protein 3 (ANGPTL3), Serum amyloid P-component (APCS), Apolipoprotein A-II (APOA2), Apolipoprotein B-100 (APOB), Apolipoprotein C3 (APOC3), Apolipoprotein C—IV (APOC4), Apolipoprotein F (APOF), Beta-2-glycoprotein 1 (APOH), Aquaporin-9 (AQP9), Bile acid-CoA: amino acid N-acyltransferase (BAAT), C4b-binding protein beta chain (C4BPB), Putative uncharacterized protein encoded by LINC01554 (C5orf27), Complement factor 3 (C3), Complement Factor 5 (C5), Complement component C6 (C6), Complement component C8 alpha chain (C8A), Complement component C8 beta chain (C8B), Complement component C8 gamma chain (C8G), Complement component C9 (C9), Calmodulin Binding Transcription Activator 1 (CAMTA1), CD38 (CD38), Complement Factor B (CFB), Complement factor H-related protein 1 (CFHR1), Complement factor H-related protein 2 (CFHR2), Complement factor H-related protein 3 (CFHR3), Cannabinoid receptor 1 (CNR1), ceruloplasmin (CP), carboxypeptidase B2 (CPB2), Connective tissue growth factor (CTGF), C—X—C motif chemokine 2 (CXCL2), Cytochro e P450 1 A2 (CYP1A2), Cytochrome P450 2A6 (CYP2A6), Cytochrome P450 2C8 (CYP2C8), Cytochrome P450 2C9 (CYP2C9), Cytochrome P450 Family 2 Subfamily D Member 6 (CYP2D6), Cytochrome P450 2E1 (CYP2E1), Phylloquinone omega-hydroxylase CYP4F2 (CYP4F2), 7-alpha-hydroxycholest-4-en-3-one 12-alpha-hydroxylase (CYP8B1), Dipeptidyl peptidase 4 (DPP4), coagulation factor 12 (F12), coagulation factor II (thrombin) (F2), coagulation factor LX (F9), fibrinogen alpha chain (FGA), fibrinogen beta chain (FGB), fibrinogen gamma chain (FGG), fibrinogen-like 1 (FGL1), flavin containing monooxygenase 3 (FM03), flavin containing monooxygenase 5 (FM05), group-specific component (vitamin D binding protein) (GC), Growth hormone receptor (GHR), glycine N-methyltransferase (GNMT), hyaluronan binding protein 2 (HABP2), hepcidin antimicrobial peptide (HAMP), hydroxyacid oxidase (glycolate oxidase) 1 (HAOl), HGF activator (HGFAC), haptoglobin-related protein; haptoglobin (HPR), hemopexin (HPX), histidine-rich glycoprotein (HRG), hydroxysteroid (11-beta) dehydrogenase 1 (HSD11B1), hydroxysteroid (17-beta) dehydrogenase 13 (HSD17B13), Inter-alpha-trypsin inhibitor heavy chain H1 (ITIH1), Inter-alpha-trypsin inhibitor heavy chain H2 (ITIH2), Inter alpha-trypsin inhibitor heavy chain H3 (ITIH3), Inter-alpha-trypsin inhibitor heavy chain H4 (ITIH4), Prekallikrein (KLKB1), Lactate dehydrogenase A (LDHA), liver expressed antimicrobial peptide 2 (LEAP2), leukocyte cell-derived chemotaxin 2 (LECT2), Lipoprotein (a) (LPA), mannan-binding lectin serine peptidase 2 (MASP2), S-adenosylmethionine synthase isoform type-1 (MAT1 A), NADPH Oxidase 4 (NOX4), Poly [ADP-ribose]polymerase 1 (PARP1), paraoxonase 1 (PON1), paraoxonase 3 (PON3), Vitamin K-dependent protein C (PROC), Retinol dehydrogenase 16 (RDH16), serum amyloid A4, constitutive (SAA4), serine dehydratase (SDS), Serpin Family A Member 1 (SERPINA1), Serpin Al 1 (SERPINA11), Kallistatin (SERPINA4), Corticosteroid-binding globulin (SERPINA6), Antithrombin-PI (SERPINC1), Heparin cofactor 2 (SERPIND1), Serpin Family H Member 1 (SERPINH1), Solute carrier family 5 member 2 (SLC5A2), Sodium/bile acid cotransporter (SLC10A1), Solute carrier family 13 member 5 (SLC13A5), Solute carrier family 22 member 1 (SLC22A1), Solute carrier family 25 member 47 (SLC25 A47), Solute carrier family 2, facilitated glucose transporter member 2 (SLC2A2), Sodium-coupled neutral amino acid transporter 4 (SLC38A4), Solute carrier organic anion transporter family member 1B1 (SLCOlB1), Sphingomyelin Phosphodiesterase 1 (SMPD1), Bile salt sulfotransferase (SEILT2A1), tyrosine aminotransferase (TAT), tryptophan 2,3-dioxygenase (TDO2), UDP glucuronosyltransferase 2 family, polypeptide B10 (UGT2B10), UDP glucuronosyltransferase 2 family, polypeptide B15 (UGT2B15), UDP glucuronosyltransferase 2 family, polypeptide B4 (UGT2B4), and vitronectin (VTN).

C. aiRNA

Like siRNA, asymmetrical interfering RNA (aiRNA) can recruit the RNA-induced silencing complex (RISC) and lead to effective silencing of a variety of genes in mammalian cells by mediating sequence-specific cleavage of the target sequence between nucleotide 10 and 11 relative to the 5′ end of the antisense strand (Sun et al., Nat. Biotech., 26:1379-1382 (2008)). Typically, an aiRNA molecule comprises a short RNA duplex having a sense strand and an antisense strand, wherein the duplex contains overhangs at the 3′ and 5′ ends of the antisense strand. The aiRNA is generally asymmetric because the sense strand is shorter on both ends when compared to the complementary antisense strand. In some aspects, aiRNA molecules may be designed, synthesized, and annealed under conditions similar to those used for siRNA molecules. As a non-limiting example, aiRNA sequences may be selected and generated using the methods described above for selecting siRNA sequences.

In another embodiment, aiRNA duplexes of various lengths (e.g., about 10-25, 12-20, 12-19, 12-18, 13-17, or 14-17 base pairs, more typically 12, 13, 14, 15, 16, 17, 18, 19, or 20 base pairs) may be designed with overhangs at the 3′ and 5′ ends of the antisense strand to target an mRNA of interest. In certain instances, the sense strand of the aiRNA molecule is about 10-25, 12-20, 12-19, 12-18, 13-17, or 14-17 nucleotides in length, more typically 12, 13, 14, 15, 16, 17, 18, 19, or 20 nucleotides in length. In certain other instances, the antisense strand of the aiRNA molecule is about 15-60, 15-50, or 15-40 nucleotides in length, more typically about 15-30, 15-25, or 19-25 nucleotides in length, and is preferably about 20-24, 21-22, or 21-23 nucleotides in length.

In some embodiments, the 5′ antisense overhang contains one, two, three, four, or more nontargeting nucleotides (e.g., “AA”, “UU”, “dTdT”, etc.). In other embodiments, the 3′ antisense overhang contains one, two, three, four, or more nontargeting nucleotides (e.g., “AA”, “UU”, “dTdT”, etc.). In certain aspects, the aiRNA molecules described herein may comprise one or more modified nucleotides, e.g., in the double-stranded (duplex) region and/or in the antisense overhangs. As a non-limiting example, aiRNA sequences may comprise one or more of the modified nucleotides described above for siRNA sequences.

In certain embodiments, aiRNA molecules may comprise an antisense strand which corresponds to the antisense strand of an siRNA molecule, e.g., one of the siRNA molecules described herein. In other embodiments, aiRNA molecules may be used to silence the expression of any of the target genes set forth above in the context of siRNA molecules, such as, e.g., genes associated with viral infection and survival, genes associated with metabolic diseases and disorders, genes associated with tumorigenesis and cell transformation, angiogenic genes, immunomodulator genes such as those associated with inflammatory and autoimmune responses, ligand receptor genes, and genes associated with neurodegenerative disorders. As a non-limiting example, the antisense oligonucleotide can be used to silence the expression of a SARS-CoV-2 gene encoding S (spike) protein, E (envelope) protein, M (membrane) protein, or N (nucleocapsid) protein.

D. miRNA

Generally, microRNAs (miRNA) are single-stranded RNA molecules of about 21-23 nucleotides in length which regulate gene expression. miRNAs are encoded by genes from whose DNA they are transcribed, but miRNAs are not translated into protein (non-coding RNA); instead, each primary transcript (a pri-miRNA) is processed into a short stem-loop structure called a pre-miRNA and finally into a functional mature miRNA. Mature miRNA molecules are either partially or completely complementary to one or more messenger RNA (mRNA) molecules, and their main function is to downregulate gene expression.

The genes encoding miRNA are much longer than the processed mature miRNA molecule. miRNA are first transcribed as primary transcripts or pri-miRNA with a cap and poly-A tail and processed to short, ˜70-nucleotide stem-loop structures known as pre-miRNA in the cell nucleus. This processing is performed in animals by a protein complex known as the Microprocessor complex, consisting of the nuclease Drosha and the double-stranded RNA binding protein Pasha (Denli et al., Nature, 432:231-235 (2004)). These pre-miRNA are then processed to mature miRNA in the cytoplasm by interaction with the endonuclease Dicer, which also initiates the formation of the RNA-induced silencing complex (RISC) (Bernstein et al., Nature, 409:363-366 (2001). Either the sense strand or antisense strand of DNA can function as templates to give rise to miRNA.

When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end (Preall et al., Curr. Biol., 16:530-535 (2006)). The remaining strand, known as the anti-guide or passenger strand, is degraded as a RISC complex substrate (Gregory et al., Cell, 123:631-640 (2005)). After integration into the active RISC complex, miRNAs base pair with their complementary mRNA molecules and induce target mRNA degradation and/or translational silencing.

Mammalian miRNA molecules are usually complementary to a site in the 3′ UTR of the target mRNA sequence. In certain instances, the annealing of the miRNA to the target mRNA inhibits protein translation by blocking the protein translation machinery. In certain other instances, the annealing of the miRNA to the target mRNA facilitates the cleavage and degradation of the target mRNA through a process similar to RNA interference (RNAi). miRNA may also target methylation of genomic sites which correspond to targeted mRNA. Generally, miRNA function in association with a complement of proteins collectively termed the miRNP.

In certain aspects, the miRNA molecules described herein are about 15-100, 15-90, 15-80, 15-75, 15-70, 15-60, 15-50, or 15-40 nucleotides in length, more typically about 15-30, 15-25, or 19-25 nucleotides in length, and are preferably about 20-24, 21-22, or 21-23 nucleotides in length. In certain other aspects, miRNA molecules may comprise one or more modified nucleotides. As a non-limiting example, miRNA sequences may comprise one or more of the modified nucleotides described above for siRNA sequences.

In some embodiments, miRNA molecules may be used to silence the expression of any of the target genes set forth above in the context of siRNA molecules, such as, e.g., genes associated with viral infection and survival, genes associated with metabolic diseases and disorders, genes associated with tumorigenesis and cell transformation, angiogenic genes, immunomodulator genes such as those associated with inflammatory and autoimmune responses, ligand receptor genes, and genes associated with neurodegenerative disorders. As a non-limiting example, the miRNA can be used to silence the expression of a SARS-CoV-2 gene encoding S (spike) protein, E (envelope) protein, M (membrane) protein, or N (nucleocapsid) protein.

In other embodiments, one or more agents that block the activity of a miRNA targeting an mRNA of interest are administered using the lipid particles described herein. Examples of blocking agents include, but are not limited to, steric blocking oligonucleotides, locked nucleic acid oligonucleotides, and Morpholino oligonucleotides. Such blocking agents may bind directly to the miRNA or to the miRNA binding site on the target mRNA.

### E. Antisense Oligonucleotides

In some embodiments, the nucleic acid component of the lipid particles described herein is an antisense oligonucleotide directed to a target gene or sequence of interest. The terms “antisense oligonucleotide” or “antisense” include oligonucleotides that are complementary to a targeted polynucleotide sequence. Antisense oligonucleotides are single strands of DNA or RNA that are complementary to a chosen sequence. Antisense RNA oligonucleotides prevent the translation of complementary RNA strands by binding to the RNA. Antisense DNA oligonucleotides can be used to target a specific, complementary (coding or non-coding) RNA. If binding occurs, this DNA/RNA hybrid can be degraded by the enzyme RNase H. In certain embodiments, antisense oligonucleotides comprise from about 10 to about 60 nucleotides or from about 15 to about 30 nucleotides. The term also encompasses antisense oligonucleotides that may not be exactly complementary to the desired target gene. Thus, the lipid particles described herein can be utilized in instances where non-target specific-activities are found with antisense, or where an antisense sequence containing one or more mismatches with the target sequence is the most preferred for a particular use. An antisense oligonucleotide can contain natural nucleotides, as well as non-natural or modified nucleotides (e.g., a modified nucleobase, modified internucleoside linkage, and/or modified sugar such as those described herein).

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tₘ, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res., 25:3389-402 (1997)).

In some embodiments, the antisense oligonucleotide component of the lipid particles described herein can be used to inhibit the expression or replication of a gene of interest. Genes of interest are set forth above in the context of siRNA molecules and include, but are not limited to, genes associated with viral infection and survival, genes associated with metabolic diseases and disorders (e.g., liver diseases and disorders), genes associated with tumorigenesis and cell transformation (e.g., cancer), angiogenic genes, immunomodulator genes such as those associated with inflammatory and autoimmune responses, ligand receptor genes, and genes associated with neurodegenerative disorders. As a non-limiting example, the antisense oligonucleotide can hybridize to a SARS-CoV-2 gene (e.g., orflab, S gene, ORF3a, E gene, M gene, ORF6, ORF7a, ORF8, N gene, or ORF10) and inhibit the expression or replication of the gene.

### F. Ribozymes

In some embodiments, the nucleic acid component of the lipid particles described herein is a ribozyme. Ribozymes are RNA-protein complexes having specific catalytic domains that possess endonuclease activity. For example, a large number of ribozymes accelerate phosphoester transfer reactions with a high degree of specificity, often cleaving only one of several phosphoesters in an oligonucleotide substrate. This specificity has been attributed to the requirement that the substrate bind via specific base-pairing interactions to the internal guide sequence (“IGS”) of the ribozyme prior to chemical reaction.

At least six basic varieties of naturally-occurring enzymatic RNA molecules are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, hepatitis δ virus, group I intron or RNaseP RNA (in association with an RNA guide sequence), or Neurospora VS RNA motif, for example. Specific examples of hammerhead motifs are described in, e.g., Rossi et al., Nucleic Acids Res., 20:4559-65 (1992). Examples of hairpin motifs are described in, e.g., EP 0360257, Hampel et al., Biochemistry, 28:4929-33 (1989); Hampel et al., Nucleic Acids Res., 18:299-304 (1990); and U.S. Pat. No. 5,631,359. An example of the hepatitis δ virus motif is described in, e.g., Perrotta et al., Biochemistry, 31:11843-52 (1992). An example of the RNaseP motif is described in, e.g., Guerrier-Takada et al., Cell, 35:849-57 (1983). Examples of the Neurospora VS RNA ribozyme motif is described in, e.g., Saville et al., Cell, 61:685-96 (1990); Saville et al., Proc. Natl. Acad. Sci. USA, 88:8826-30 (1991); Collins et al., Biochemistry, 32:2795-9 (1993). An example of the Group I intron is described in, e.g., U.S. Pat. No. 4,987,071. Important characteristics of enzymatic nucleic acid molecules are that they have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein.

Methods of producing a ribozyme targeted to any polynucleotide sequence are known in the art. Ribozymes may be designed as described in, e.g., PCT Publication Nos. WO 93/23569 and WO 94/02595, and synthesized to be tested in vitro and/or in vivo as described therein.

Ribozyme activity can be optimized by altering the length of the ribozyme binding arms or chemically synthesizing ribozymes with modifications that prevent their degradation by serum ribonucleases (see, e.g., PCT Publication Nos. WO 92/07065, WO 93/15187, WO 91/03162, and WO 94/13688; EP 92110298.4; and U.S. Pat. No. 5,334,711, which describe various chemical modifications that can be made to the sugar moieties of enzymatic RNA molecules), modifications which enhance their efficacy in cells, and removal of stem II bases to shorten RNA synthesis times and reduce chemical requirements.

### G. Immunostimulatory Oligonucleotides

In some embodiments, the nucleic acid component of the lipid particles described herein is an immunostimulatory oligonucleotide (ISS; single- or double-stranded) capable of inducing an immune response when administered to a subject, which may be a mammal such as a human. ISS include, e.g., certain palindromes leading to hairpin secondary structures (see, Yamamoto et al., J. Immunol., 148:4072-6 (1992)), or CpG motifs, as well as other known ISS features (such as multi-G domains; see; PCT Publication No. WO 96/11266).

Immunostimulatory nucleic acids are considered to be non-sequence specific when it is not required that they specifically bind to and reduce the expression of a target sequence in order to provoke an immune response. Thus, certain immunostimulatory nucleic acids may comprise a sequence corresponding to a region of a naturally-occurring gene or mRNA, but they may still be considered non-sequence specific immunostimulatory nucleic acids.

In one embodiment, the immunostimulatory nucleic acid or oligonucleotide comprises at least one CpG dinucleotide. The oligonucleotide or CpG dinucleotide may be unmethylated or methylated. In another embodiment, the immunostimulatory nucleic acid comprises at least one CpG dinucleotide having a methylated cytosine. In one embodiment, the nucleic acid comprises a single CpG dinucleotide, wherein the cytosine in the CpG dinucleotide is methylated. In an alternative embodiment, the nucleic acid comprises at least two CpG dinucleotides, wherein at least one cytosine in the CpG dinucleotides is methylated. In a further embodiment, each cytosine in the CpG dinucleotides present in the sequence is methylated. In another embodiment, the nucleic acid comprises a plurality of CpG dinucleotides, wherein at least one of the CpG dinucleotides comprises a methylated cytosine. In certain embodiments, the oligonucleotides used in the compositions and methods described herein have a phosphodiester (“PO”) backbone or a phosphorothioate (“PS”) backbone, and/or at least one methylated cytosine residue in a CpG motif.

### VI. Preparation of Lipid Particles

The lipid particles described herein, in which a nucleic acid such as an mRNA may be encapsulated and protected from degradation, can be formed by any method known in the art including, but not limited to, a continuous mixing method and a direct dilution process.

In some embodiments, the lipid particles described herein are produced via a continuous mixing method, e.g., a process that includes providing an aqueous solution comprising a nucleic acid in a first reservoir, providing an organic lipid solution in a second reservoir, and mixing the aqueous solution with the organic lipid solution such that the organic lipid solution mixes with the aqueous solution so as to substantially instantaneously produce a lipid particle encapsulating the nucleic acid. This process and the apparatus for carrying this process are described in U.S. Patent Publication No. 20040142025.

The action of continuously introducing lipid and buffer solutions into a mixing environment, such as in a mixing chamber, causes a continuous dilution of the lipid solution with the buffer solution, thereby producing a lipid particle substantially instantaneously upon mixing. As used herein, the phrase “continuously diluting a lipid solution with a buffer solution” (and variations) generally means that the lipid solution is diluted sufficiently rapidly in a hydration process with sufficient force to effectuate vesicle generation. By mixing the aqueous solution comprising a nucleic acid with the organic lipid solution, the organic lipid solution undergoes a continuous stepwise dilution in the presence of the buffer solution (i.e., aqueous solution) to produce a lipid particle.

The lipid particles formed using the continuous mixing method typically have a size of from about 40 nm to about 150 nm, from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

In some embodiments, the lipid particles described herein are produced via a direct dilution process that includes forming a lipid particle solution and immediately and directly introducing the lipid particle solution into a collection vessel containing a controlled amount of dilution buffer. In certain embodiments, the collection vessel includes one or more elements configured to stir the contents of the collection vessel to facilitate dilution. In one embodiment, the amount of dilution buffer present in the collection vessel is substantially equal to the volume of lipid particle solution introduced thereto. As a non-limiting example, a lipid particle solution in 45% ethanol when introduced into the collection vessel containing an equal volume of dilution buffer will advantageously yield smaller particles.

In some embodiments, the lipid particles described herein are produced via a direct dilution process in which a third reservoir containing dilution buffer is fluidly coupled to a second mixing region. In this embodiment, the lipid particle solution formed in a first mixing region is immediately and directly mixed with dilution buffer in the second mixing region. In certain embodiments, the second mixing region includes a T-connector arranged so that the lipid particle solution and the dilution buffer flows meet as opposing 1800 flows; however, connectors providing shallower angles can be used, e.g., from about 27° to about 180°. A pump mechanism delivers a controllable flow of buffer to the second mixing region. In one embodiment, the flow rate of dilution buffer provided to the second mixing region is controlled to be substantially equal to the flow rate of lipid particle solution introduced thereto from the first mixing region. This embodiment advantageously allows for more control of the flow of dilution buffer mixing with the lipid particle solution in the second mixing region, and therefore also the concentration of lipid particle solution in buffer throughout the second mixing process. Such control of the dilution buffer flow rate advantageously allows for small particle size formation at reduced concentrations.

These processes and the apparatuses for carrying out these direct dilution processes are described in U.S. Patent Publication No. 20070042031.

The lipid particles formed using the direct dilution process typically have a size of from about 40 nm to about 150 nm, from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

If needed, the lipid particles described herein can be sized by any of the methods available to one of skill in the art. The sizing may be conducted in order to achieve a desired size range and relatively narrow distribution of particle sizes.

Several techniques are available for sizing the particles to a desired size. One sizing method, used for liposomes and equally applicable to the present particles, is described in U.S. Pat. No. 4,737,323. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and about 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In some embodiments, the nucleic acid to lipid ratios (mass/mass ratios) in a formed lipid particle ranges from about 0.01 to about 0.2, from about 0.02 to about 0.1, from about 0.03 to about 0.1, or from about 0.01 to about 0.08. The ratio of the starting materials also falls within this range. In other embodiments, the lipid particle preparation uses about 400 μg nucleic acid per 10 mg total lipid or a nucleic acid to lipid mass ratio of about 0.01 to about 0.08, e.g., about 0.04, which corresponds to 1.25 mg of total lipid per 50 μg of nucleic acid. In certain embodiments, the particle has a nucleic acid:lipid mass ratio of about 0.08.

In other embodiments, the lipid to nucleic acid ratios (mass/mass ratios) in a formed lipid particle ranges from about 1 (1:1) to about 100 (100:1), from about 5 (5:1) to about 100 (100:1), from about 1 (1:1) to about 50 (50:1), from about 2 (2:1) to about 50 (50:1), from about 3 (3:1) to about 50 (50:1), from about 4 (4:1) to about 50 (50:1), from about 5 (5:1) to about 50 (50:1), from about 1 (1:1) to about 25 (25:1), from about 2 (2:1) to about 25 (25:1), from about 3 (3:1) to about 25 (25:1), from about 4 (4:1) to about 25 (25:1), from about 5 (5:1) to about 25 (25:1), from about 5 (5:1) to about 20 (20:1), from about 5 (5:1) to about 15 (15:1), from about 5 (5:1) to about 10 (10:1), about 5 (5:1), 6 (6:1), 7 (7:1), 8 (8:1), 9 (9:1), 10 (10:1), 11 (11:1), 12 (12:1), 13 (13:1), 14 (14:1), or 15 (15:1). The ratio of the starting materials also falls within this range.

### VII. Administration of Lipid Particles

Once formed, the lipid particles described herein are useful for the introduction of nucleic acids such as mRNA into cells. Accordingly, the present disclosure also provides methods for introducing a nucleic acid such as an mRNA into a cell. The methods are carried out in vitro or in vivo by first forming the particles and then contacting the particles with the cells for a period of time sufficient for delivery of the nucleic acid to the cells to occur.

The lipid particles described herein can be adsorbed to almost any cell type with which they are mixed or contacted. Once adsorbed, the particles can either be endocytosed by a portion of the cells, exchange lipids with cell membranes, or fuse with the cells. Transfer or incorporation of the nucleic acid portion of the particle can take place via any one of these pathways. In particular, when fusion takes place, the particle membrane is integrated into the cell membrane and the contents of the particle combine with the intracellular fluid.

The lipid particles described herein can be administered either alone or in a mixture with a pharmaceutically acceptable carrier (e.g., physiological saline or phosphate buffer) selected in accordance with the route of administration and standard pharmaceutical practice. Generally, normal buffered saline (e.g., 135-150 mM NaCl) is used as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. Additional suitable carriers are described in, e.g., REMINGTON'S PHARMACEUTICAL SCIENCES, Mack Publishing Company, Philadelphia, PA, 17th ed. (1985). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The phrase “pharmaceutically acceptable” refers to molecular entities and compositions that do not produce an allergic or similar untoward reaction when administered to a human.

The pharmaceutically acceptable carrier is generally added following particle formation. Thus, after the particle is formed, the particle can be diluted into pharmaceutically acceptable carriers such as normal buffered saline.

The concentration of particles in the pharmaceutical formulations can vary widely, e.g., from less than about 0.05%, usually at or at least about 2 to about 5%, to as much as about 10 to about 90% by weight, and can be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

The pharmaceutical compositions described herein may be sterilized by conventional, well-known sterilization techniques. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

### A. In Vivo Administration

In some embodiments, the lipid particles described herein are administered to a subject by systemic delivery, e.g., to a distal target cell via body systems such as the circulation. In certain embodiments, the present disclosure provides fully encapsulated lipid particles that protect the nucleic acid from nuclease degradation in serum, are nonimmunogenic, are small in size, and are suitable for repeat dosing.

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol., 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993). Still other methods of administering lipid-based therapeutics are described in, for example, U.S. Pat. Nos. 3,993,754; 4,145,410; 4,235,871; 4,224,179; 4,522,803; and 4,588,578. The lipid particles can be administered by direct injection at the site of disease or by injection at a site distal from the site of disease (see, e.g., Culver, HUMAN GENE THERAPY, MaryAnn Liebert, Inc., Publishers, New York. pp. 70-71(1994)).

The compositions described herein, either alone or in combination with other suitable components, can be made into aerosol formulations (i.e., they can be “nebulized”) to be administered via inhalation (e.g., intranasally or intratracheally) (see, Brigham et al., Am. J. Sci., 298:278 (1989)). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In particular embodiments, the compositions are administered intravenously (e.g., by intravenous infusion), intramuscularly, pulmonarily, orally, topically, intranasally, intracerebrally, intraperitoneally, intravesically, or intrathecally.

Generally, when administered intravenously, the lipid particle formulations are formulated with a suitable pharmaceutical carrier. Many pharmaceutically acceptable carriers may be employed in the compositions and methods described herein. Suitable formulations for use are found, for example, in REMINGTON'S PHARMACEUTICAL SCIENCES, Mack Publishing Company, Philadelphia, PA, 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) is used as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

In certain applications, the lipid particles described herein may be delivered via oral administration to a subject. The particles may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451). These oral dosage forms may also contain binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

Typically, these oral formulations may contain at least about 0.1% of the lipid particles or more, although the percentage of the particles may, of course, be varied and may conveniently be between about 1% or about 2% and about 60% or about 70% or more of the weight or volume of the total formulation. Naturally, the amount of particles in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Formulations suitable for oral administration can consist of: (a) liquid solutions, such as an effective amount of lipid particles comprising nucleic acid (e.g., mRNA) suspended in diluents such as water, saline, or PEG 400; (b) capsules, sachets, or tablets, each containing a predetermined amount of lipid particles comprising nucleic acid (e.g., mRNA), as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise lipid particles comprising nucleic acid (e.g., mRNA) in a flavor, e.g., sucrose, as well as pastilles comprising the lipid particles in an inert base, such as gelatin and glycerin or sucrose and acacia emulsions, gels, and the like containing, in addition to the lipid particles, carriers known in the art.

In another example of their use, lipid particles can be incorporated into a broad range of topical dosage forms. For instance, a suspension containing lipid particles can be formulated and administered as gels, oils, emulsions, topical creams, pastes, ointments, lotions, foams, mousses, and the like.

When preparing pharmaceutical preparations of the lipid particles described herein, it is preferable to use quantities of the particles which have been purified to reduce or eliminate empty particles or particles with nucleic acid associated with the external surface.

The methods described herein may be practiced in a variety of hosts. Preferred hosts include mammalian species, such as primates (e.g., humans and chimpanzees as well as other nonhuman primates), canines, felines, equines, bovines, ovines, caprines, rodents (e.g., rats and mice), lagomorphs, and swine.

The amount of particles administered will depend upon the ratio of nucleic acid (e.g., mRNA) to lipid, the particular nucleic acid used, the disease or disorder being treated, the age, weight, and condition of the subject, and the judgment of the clinician, but will generally be between about 0.01 mg/kg and about 50 mg/kg of body weight, between about 0.1 mg/kg and about 5 mg/kg of body weight, or about 10⁸-10¹⁰ particles per administration (e.g., injection).

B. In vitro Administration

For in vitro applications, the delivery of nucleic acids (e.g., mRNA) can be to any cell grown in culture, whether of plant or animal origin, vertebrate or invertebrate, and of any tissue or type. In certain embodiments, the cells are animal cells, e.g., mammalian cells such as human cells.

Contact between the cells and the lipid particles, when carried out in vitro, generally takes place in a biologically compatible medium. The concentration of particles can vary widely depending on the particular application, but is generally between about 1 mol and about 10 mmol. Treatment of the cells with the lipid particles is generally carried out at physiological temperatures (about 37° C.) for periods of time ranging from about 1 to about 48 hours, e.g., from about 2 to about 4 hours.

In some embodiments, a lipid particle suspension is added to 60-80% confluent plated cells having a cell density of from about 10³ to about 10⁵ cells/ml, e.g., about 2×10⁴ cells/ml. The concentration of the suspension added to the cells can be from about 0.01 to 0.2 g/ml, e.g., about 0.1 g/ml.

Using an Endosomal Release Parameter (ERP) assay, the delivery efficiency of the lipid particle can be optimized. An ERP assay is described in U.S. Patent Publication No. 20030077829. More particularly, the purpose of an ERP assay is to distinguish the effect of various cationic lipids and helper lipid components based on their relative effect on binding/uptake or fusion with/destabilization of the endosomal membrane. This assay allows one to determine quantitatively how each component of the lipid particle affects delivery efficiency, thereby optimizing the lipid particle. Usually, an ERP assay measures expression of a reporter protein (e.g., luciferase, 0-galactosidase, green fluorescent protein (GFP), etc.), and in some instances, a lipid particle formulation optimized for an expression plasmid will also be appropriate for encapsulating other types of nucleic acid such as mRNA. In some instances, an ERP assay can be adapted to measure downregulation of transcription or translation of a target sequence in the presence or absence of an interfering RNA (e.g., siRNA). In other instances, an ERP assay can be adapted to measure the expression of a target protein in the presence or absence of an mRNA. By comparing the ERPs for each of the various lipid particles, one can readily determine the optimized system, e.g., the lipid particle that has the greatest uptake in the cell.

### C. Cells for Delivery of Lipid Particles

The compositions and methods described herein are used to treat a wide variety of cell types, in vivo and in vitro. Suitable cells include, e.g., hematopoietic precursor (stem) cells, fibroblasts, keratinocytes, hepatocytes, immune cells, endothelial cells, skeletal and smooth muscle cells, osteoblasts, neurons, quiescent lymphocytes, terminally differentiated cells, slow or noncycling primary cells, parenchymal cells, lymphoid cells, epithelial cells, bone cells, and the like. In some embodiments, lipid particles comprising nucleic acid (e.g., mRNA) are delivered to immune cells such as e.g., antigen-presenting cells (e.g., dendritic cells, macrophages, B cells) and T cells (e.g., helper T cells, cytotoxic T cells, memory T cells, regulatory T cells, natural killer T cells). In some embodiments, lipid particles comprising nucleic acid (e.g., siRNA) are delivered to cancer cells such as, e.g., lung cancer cells, colon cancer cells, rectal cancer cells, anal cancer cells, bile duct cancer cells, small intestine cancer cells, stomach (gastric) cancer cells, esophageal cancer cells, gallbladder cancer cells, liver cancer cells, pancreatic cancer cells, appendix cancer cells, breast cancer cells, ovarian cancer cells, cervical cancer cells, prostate cancer cells, renal cancer cells, cancer cells of the central nervous system, glioblastoma tumor cells, skin cancer cells, lymphoma cells, choriocarcinoma tumor cells, head and neck cancer cells, osteogenic sarcoma tumor cells, and blood cancer cells.

In vivo delivery of lipid particles comprising nucleic acid is suited for targeting cells of any cell type. The methods and compositions can be employed with cells of a wide variety of vertebrates, including mammals, such as, e.g., canines, felines, equines, bovines, ovines, caprines, rodents (e.g., mice, rats, and guinea pigs), lagomorphs, swine, and primates (e.g., monkeys, chimpanzees, and humans).

### EXAMPLES

The present disclosure will be described in greater detail by way of specific examples. The following examples are offered for illustrative purposes only, and are not intended to limit the disclosure in any manner.

### Example 1. Synthesis of Ditetradecylamine for Poly(Alkyloxazoline)-Lipid Conjugates

Ditetradecylamine (5) was synthesized according to the scheme shown below.

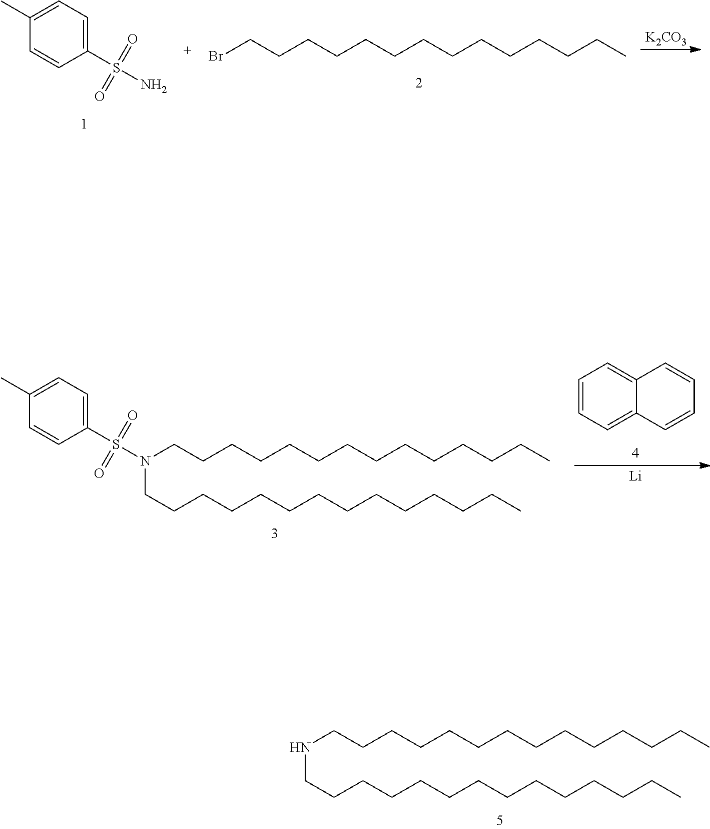

Synthesis of 4-methyl-N,N-ditetradecylbenzenesulfonamide (3). K₂CO₃ (20.18 g, 146.0 mmol), p-toluenesulfonamide (1) (5.00 g, 29.2 mmol) and 1-bromotetradecane (2) (21.7 ml, 73.0 mmol) were heated at reflux in DMF (75 ml) for 20 h. The reaction was cooled to RT, diluted with Et₂O (100 ml) and filtered. The filtrate was washed with H₂O (50 ml) and the aqueous back extracted with Et₂O (100 ml). The combined organics were dried (Na₂SO₄), filtered, concentrated in-vacuo and the residue purified by automated flash chromatography (DCM/hexane 40/60) to give 4-methyl N,N-ditetradecyl-p-toluenesulfonamide (3) (15.86 g, 96%).

Synthesis of ditetradecylamine (5). 4-Methyl-N,N-ditetradecylbenzenesulfonamide (3) (10 g, 17.7 mmol) in THE (5 ml) was added to a solution of lithium naphthalenide (prepared from stirring naphthalene (4) (11.36 g, 88.7 mmol) and lithium metal (0.92 g, 133.0 mmol) in THE (40 ml) at RT for 1 h) at 0° C. and the reaction stirred for 1 hr at RT. MeOH (4 ml) was added, followed by H₂O (50 ml) and the mixture extracted with ether (100 ml). The organics were dried (Na₂SO₄), filtered and concentrated in-vacuo. The residue was purified by automated flash chromatography (EtOAc/Hex (50:50)) to give ditetradecylamine (5) (4.85 g, 66.7%).

### Example 2. Synthesis of 2,3-Bis(Tetradecyloxy)Propan-1-Amine for Poly(Alkyloxazoline)-Lipid Conjugates

2,3-bis(Tetradecyloxy)propan-1-amine (TFA salt) (10) was synthesized according to the following scheme.

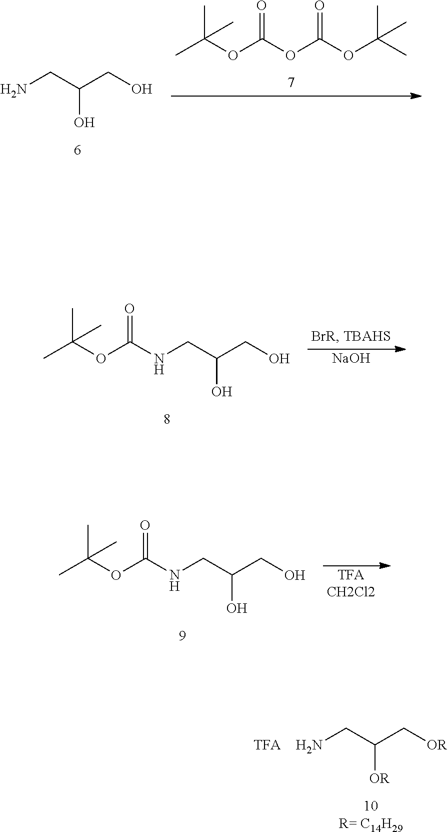

Synthesis of tert-butyl (2,3-dihydroxypropyl)carbamate (8). Di-t-butyl dicarbonate (7) (14.23 g, 65.2 mmol) and 3-amino-1,2-propanediol (6) (5.4 g, 59.3 mmol) were stirred in DCM/MeOH (1:1) at RT for 16 hr. The reaction was concentrated in-vacuo to give 8 (11.25 g, 99%) which was used without purification.

Synthesis of tert-butyl (2,3-bis(tetradecyloxy)propyl)carbamate (9). tert-Butyl (2,3-dihydroxypropyl)carbamate (8) (5 g, 26.2 mmol), bromotetradecane (21.75 g, 78.5 mmol) and TBAHS (4.44 g, 13.1 mmol) were stirred in toluene (20 mL) at 0° C. NaOH (15 mL, 50% w/v) was added and the Bi-phasic mixture stirred vigorously for 18 hr allowing to warm to RT. The reaction was diluted with water and extracted with hexane. The combined organics were washed (brine), dried (MgSO₄), filtered and concentrated in-vacuo. The crude material was subjected to chromatography (10% EtOAc-hexane) to give tert-butyl (2,3-bis(tetradecyloxy)propyl)carbamate (9) (Quant).

Synthesis of 2,3-bis(tetradecyloxy)propan-1-amine (TFA salt) (10). tert-butyl (2,3-bis(tetradecyloxy)propyl)carbamate (9) prepared in step 2 was stirred in TFA/DCM (1:1) at RT for 2 hr. The reaction was concentrated in-vacuo to give 2,3-bis(tetradecyloxy)propan-1-amine (TFA salt) (10) (8.3 g, Quant) which was used without purification.

### Example 3. Synthesis of Poly(Alkyloxazoline)-Lipid Conjugates

N-Alkylated thiopropanamide POZ lipids were synthesized as shown below for conjugate (11).

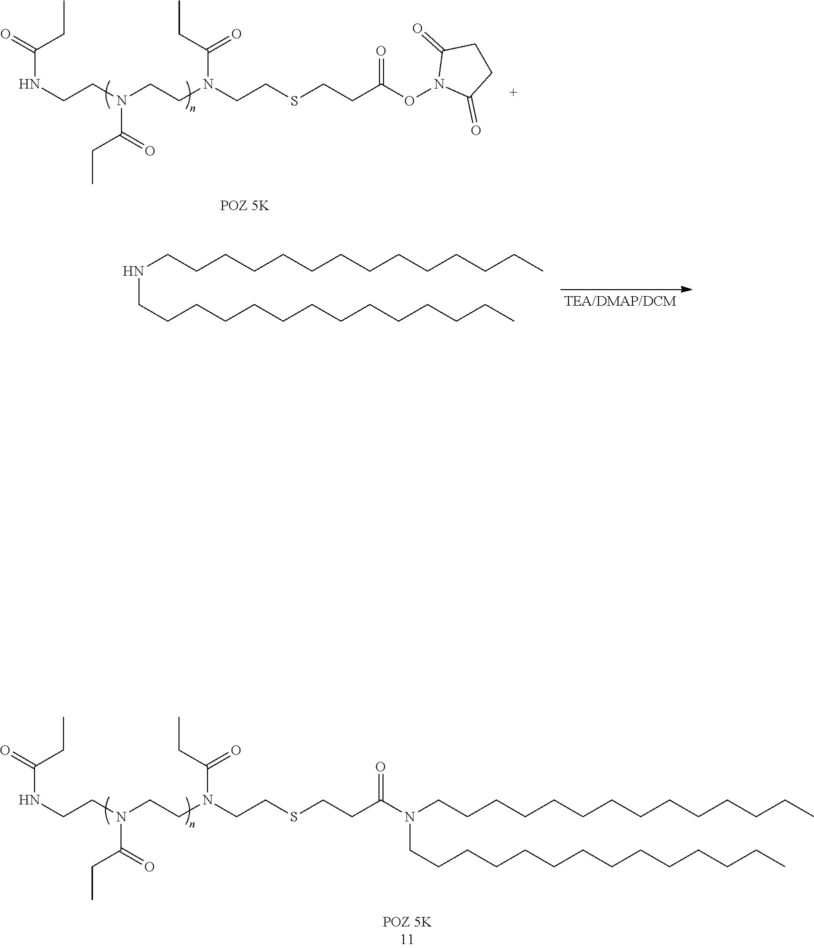

The reagents were stirred in DCM at RT for 16 hr. The reaction was concentrated in-vacuo and the residue purified by automated flash chromatography (0-25% MeOH/DCM). Similar product containing fractions were combined and concentrated in-vacuo. The residue was taken in up the minimum volume of DCM and Et₂O added to produce a precipitate which was recovered by filtration to give 11 (38 mg, 11.7%). ¹H NMR (400 MHz, CDCl3) δ 3.47 (bs, 159H), 2.46-2.26 (m, 83H), 1.76 (s, 33H), 1.3 (m, 33H), 1.14 (m, 122H), 0.90 (m, 6H).

The following lipid conjugates were prepared using analogous methodology as described above with the appropriate amine.

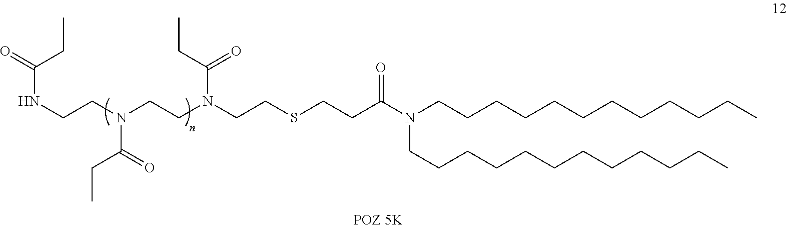

Compound (12). ¹H NMR (400 MHz, CDCl3). 3.46 (bs, 126H), 2.46-2.25 (m, 57H), 1.78 (S, 9H), 1.27 (m. 24H), 1.15 (m, 99H), 0.90 (t, J=4 Hz), 6H).

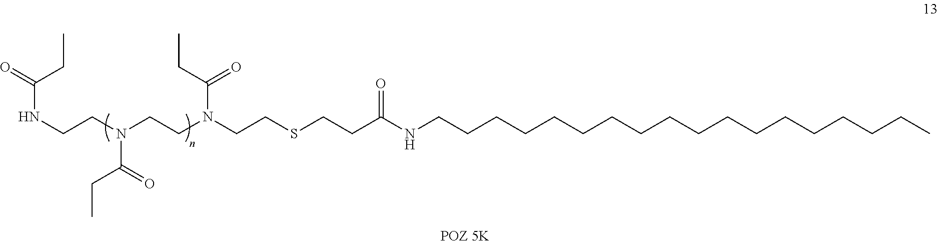

Compound (13). ¹H NMR (400 MHz, CDCl3). 3.46 (bs, 145H), 2.46-2.26 (m, 78H), 1.72 (bs, 24H), 1.25 (m, 21H), 1.14 (m, 112H), 0.9 (m, 3H).

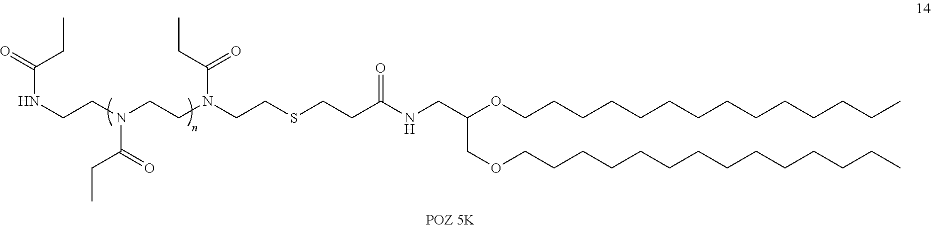

Compound (14). ¹H NMR (400 MHz, CDCl3). 3.46 (bs, 128H), 2.85 (m, 2H), (2.5-2.24) (m, 54H), 1.78 (s, 32H), 1.57 (m, 4H), 1.26 (m, 32H), 1.12 (m, 92H), 0.89 (t, J=4 Hz, 6H).

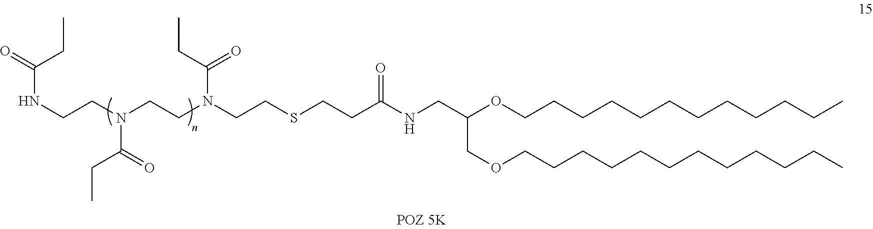

Compound (15). ¹H NMR (400 MHz, CDCl3) 3.49 (bs, 136H), 2.46-2.26 (m, 60H), 1.7 (bs, 20H), 1.57 (m, 5H), 1.27 (m, 30H), 1.14 (bs, 100H), 0.89 (t, 6H, J=4 Hz).

### Example 4. General Methods for Lipid Nanoparticle Preparation and Characterization

### Lipid Nanoparticle (LNP) Formulations

The lipid solution contains 4 components: a polymer-conjugated lipid, an ionizable cationic lipid (e.g., (6Z,16Z)-12-((Z)-dec-4-en-1-yl)docosa-6,16-dien-11-yl 5-(dimethylamino)-pentanoate), cholesterol, and a phospholipid (e.g., DSPC). Lipid stocks were prepared using the lipid identities and molar ratios as described, to achieve a total concentration of approximately 7 mg/mL in 100% ethanol. Firefly luciferase (Luc) mRNA (TriLink Biotechnologies, L-7202) or Ovalbumin (OVA) mRNA (TriLink Biotechnologies, L-7210) was diluted in acetate, pH 5 buffer and nuclease free water to achieve a target concentration of 0.366 mg/mL mRNA in 100 mM acetate, pH 5. Equal volumes of the lipid and nucleic acid solutions were blended at a flow rate of 400 mL/min through a T-connector, and diluted with approximately 4 volumes of PBS, pH 7.4. Formulations were placed in Slide-A-Lyzer dialysis units (MWCO 10,000) and dialyzed overnight against 10 mM Tris, 500 mM NaCl, pH 8 buffer. Following dialysis, the formulations were concentrated to approximately 0.5 mg/mL using VivaSpin concentrator units (MWCO 100,000) and dialyzed overnight against 5 mM Tris, 10% sucrose, pH 8 buffer. Formulations were filtered through a 0.2 μm syringe filter and stored at −80° C. until use. Nucleic acid concentration was determined by the RiboGreen assay. Particle size (hydrodynamic diameter) and polydispersity index (PDI) were determined by dynamic light scattering measurements using a Malvern Nano Series Zetasizer.

### In Vivo Testing of Lipid Nanoparticles Via Intravenous Administration (Luciferase Model)

LNP formulations encapsulating firefly luciferase were injected intravenously at 0.5 mg/kg to female BALB/c mice (6-8 weeks old). On day of injection, the LNP stocks were filtered and diluted to the required dosing concentration with phosphate buffered saline, pH 7.4. At 6 hours post-dose administration, animals were anesthetized with a lethal dosage of ketamine/xylazine. Blood samples were collected into EDTA microtainer tubes and centrifuged at 16,000×g for 5 minutes at 4° C. All plasma samples were stored at −80° C. until cytokine analysis was performed. Liver samples were collected, weighed, and flash frozen in liquid nitrogen. Liver samples were stored in FastPrep© tubes at −80° C. until analyzed for luciferase activity.

### Cytokine Analysis

The production of proinflammatory cytokines (i.e., Monocyte Chemoattractant Protein-1, MCP-1) was measured by ELISA using pair-matched monoclonal antibodies from BD Biosciences. Plates were coated with capture antibody and stored at 2-8° C. overnight. The following day, the plates were blocked with 10% FBS in 1×PBS and test samples were added. After washing three times, the detection antibody was added and incubated for 1 h. After incubation and washing, TMB substrate was added for color development and the reaction was stopped with sulfuric acid. The plates were read at 450/570 OD.

### Luciferase Activity Analysis

Frozen aliquots of liver were thawed and homogenized in 1 mL of 1×CCLR (Cell Culture Lysis Reagent) using a FastPrep© homogenizer. The homogenate was centrifuged at 16,000 rpm for 10 minutes at 4° C. Twenty (20) μL of the supernatant was loaded into a 96-well white plate and luminescence measured following addition of luciferase reagent (Promega Luciferase Assay System). Luciferase activity was determined by comparing luminescence of the homogenized samples to luciferase protein standards. To account for any quenching of luminescence by components in the liver homogenate, luciferase was added to the liver homogenates of untreated animals and the resulting luminescence was measured. A quench factor was applied to all samples to obtain the corrected luciferase activity values, and normalized per unit mass of tissue analyzed.

### In Vivo Testing of Lipid Nanoparticles Via Intramuscular Administration (Ovalbumin Immunogenicity Model)

LNP formulations encapsulating OVA mRNA were administered intramuscularly at 1 μg dose to BALB/c mice (7-8 weeks old) on Day 0 (prime) and Day 21 (boost). Blood samples were collected into EDTA microtainer tubes at pre-dose and at days 7, 14, 21, and 28. At Day 35, animals were anesthetized with a lethal dosage of ketamine/xylazine. Blood samples were collected into EDTA microtainer tubes and centrifuged at 16,000×g for 5 minutes at 4° C. All plasma samples were stored at −80° C. until anti-OVA IgG ELISA was performed. Spleen samples were also collected at the terminal timepoint and splenocytes were harvested for the ELISpot assay.

### Anti-OVA IgG Analysis

An ELISA method was used to measure the production of anti-OVA IgG antibodies. Plates were coated with ovalbumin protein and stored at 2-8° C. overnight. The following day, the plates were blocked with 10% FBS in PBS and samples were added. After washing, the secondary antibody was added and incubated for 2 hours at room temperature. Following incubation, the plates were washed and TMB substrate was added for color development. The reaction was stopped with 2 N sulfuric acid and the plates were read at 450/570 OD.

### ELISpot Analysis

At the terminal timepoint, spleen samples were collected from animals and splenocytes were isolated. ELISpot plates were prepared by washing the plates and incubating with media for at least 30 minutes. Following incubation, media was removed, and stimuli was added. Cells were added and incubated overnight at 37° C. After overnight incubation, cells were removed, and plates were washed. Detection antibody was added, followed by a 2-hour incubation at room temperature. Plates were washed and streptavidin-HRP antibody was added. Following 1-hour incubation at room temperature, plates were washed and TMB substrate was added for color development. The reaction was stopped with 2 N sulfuric acid and the plates were read at 450/570 OD.

### Example 5. Particle Characteristics of LNP Formulations Containing Poly(2-Ethyl 2-Oxazoline) (PEOZ)-Lipid Conjugates

PEOZ lipids of −5000 Da with different alkyl chain lengths and linkers were formulated into LNPs and compared to two PEG-lipid controls. Luc mRNA was used as the payload. Despite the hydrophobicity of the PEOZ 5000 polymer, its incorporation into LNP resulted in the formation of stable particles. Importantly, LNPs formulated with different PEOZ lipid chemistries reported comparable physicochemical characteristics to those prepared with PEG2000-C-DMA and the commercially available PEG-DMG lipid (Table 1).

TABLE 1
Particle Characteristics of LNP Formulations Containing
Various PEOZ Lipids

| | Final Product | |
|---|---|---|
| LNP mol ratios of 1.6:54.6:32.8:10.9, Polymer-conjugated | Z-avg | % |
| lipid:Cationic lipid:Cholesterol:DSPC | (PDI) nm | EE |
| PEG2000-C-DMA (2xC14) | 73 (0.15) | 98 |
| PEG-DMG (2xC14) | 85 (0.06) | 98 |
| Compound 14 | 74 (0.09) | 96 |
| Compound 11 | 71 (0.09) | 89 |
| Compound 12 | 71 (0.12) | 82 |
| Compound 13 | 76 (0.11) | 84 |
| Compound 15 | 71 (0.08) | 89 |

% EE = encapsulation efficiency;
PDI = polydispersity

### Example 6. Performance of LNP Containing PEOZ-Lipid Conjugates in an Intravenous Mouse Model

LNP formulations bearing a luciferase mRNA payload and PEOZ5000 lipids were compared to the benchmark controls (PEG2000-C-DMA and PEG-DMG) for activity in a luciferase mouse model following intravenous administration. At 6h post-dose, the PEOZ lipid formulations mediated high levels of luciferase activity in the liver, reporting equivalent or improved activity compared to the controls (Table 2). Furthermore, these PEOZ lipid formulations reported lower luciferase expression in the spleen compared to the commercially available PEG-DMG, indicating less non-specific (off-target) activity (Table 3).

TABLE 2
Luciferase activity in liver at 6 hours following intravenous administration of 0.5 mg/kg
LNP containing PEG-conjugated or PEOZ-conjugated lipids in BALB/c mice (n = 4)

| Treatment | 6 h post-IV administration | |
|---|---|---|
| [LNP mol ratios of 1.6:54.6:32.8:10.9, | Luc Activity | Std Dev |
| Polymer-conjugated lipid:Cationic lipid:Cholesterol:DSPC] | (pg/g liver) | (pg/g liver) |
| PEG2000-C-DMA (2xC14) | 6.1E+07 | 1.2E+07 |
| PEG-DMG (2xC14) | 5.5E+07 | 2.1E+07 |
| Compound 14 | 4.5E+07 | 7.1E+06 |
| Compound 11 | 6.4E+07 | 1.3E+07 |
| Compound 12 | 5.0E+07 | 1.6E+07 |
| Compound 13 | 4.9E+07 | 1.1E+07 |
| Compound 15 | 8.2E+07 | 2.2E+07 |

TABLE 3
Luciferase activity in spleen at 6 hours following intravenous administration of 0.5 mg/kg
LNP containing PEG-conjugated or PEOZ-conjugated lipids in BALB/c mice (n = 4)

| Treatment | 6 h post-IV administration | |
|---|---|---|
| [LNP mol ratios of 1.6:54.6:32.8:10.9, | Luc Activity | Std Dev |
| Polymer-conjugated lipid:Cationic lipid:Cholesterol:DSPC] | (pg/g spleen) | (pg/g spleen) |
| PEG2000-C-DMA (2xC14) | 6.7E+05 | 3.2E+05 |
| PEG-DMG (2xC14) | 2.1E+06 | 3.2E+05 |
| Compound 14 | 7.7E+05 | 2.1E+05 |
| Compound 11 | 5.6E+05 | 1.9E+05 |
| Compound 12 | 7.8E+05 | 1.6E+05 |
| Compound 13 | 5.9E+05 | 1.1E+05 |
| Compound 15 | 1.5E+06 | 6.1E+05 |

To assess immunostimulatory potential of these PEOZ formulations, MCP-1 induction was measured in plasma samples at 6h following intravenous administration. The PEOZ-based LNPs induced similar or lower cytokine levels compared to the established PEGylated counterparts (Table 4).

TABLE 4
MCP-1 induction at 6 hours following intravenous administration of 0.5 mg/kg Luciferase
mRNA-LNP containing PEG-conjugated or PEOZ-conjugated lipids in BALB/c mice (n = 4)

| | 6 h post-IV | |
|---|---|---|
| Treatment | administration | |
| [LNP mol ratios of 1.6:54.6:32.8:10.9, | MCP-1 | Std Dev |
| Polymer-conjugated lipid:Cationic lipid:Cholesterol:DSPC] | (pg/mL) | (pg/mL) |
| PBS | 58 | 5 |
| PEG2000-C-DMA (2xC14) | 87 | 12 |
| PEG-DMG (2xC14) | 196 | 41 |
| Compound 14 | 104 | 30 |
| Compound 11 | 73 | 8 |
| Compound 12 | 111 | 16 |
| Compound 13 | 106 | 36 |
| Compound 15 | 103 | 24 |

### Example 7. Effect of PEOZ-Lipid on Clearance Rate in Plasma

LNP formulations bearing PEOZ lipids of ˜5000 Da were compared to the benchmark control (PEG2000-C-DMA) for the rate of clearance from the plasma following intravenous injection. Ionizable lipid level in the compositions was used as a proxy to evaluate rate of clearance using mass spectrometry. At LNP ratios of 1.6:54.6:32.8:10.9, Polymer-conjugated lipid:Cationic lipid:Cholesterol:DPSC, the PEOZ-containing formulations (Compound 11, 12, 13, 14, and 15) showed improved rate of clearance from the plasma at 0.25 hours post-injection compared to the benchmark (Table 5). The shorter residence time in the plasma compartment exhibited by the PEOZ-containing LNPs could potentially help mitigate antibody responses upon repeat dose administration.

TABLE 5
Plasma rate of clearance following intravenous injection of 0.5 mg/kg Luciferase
mRNA-LNP containing PEG-conjugated or PEOZ-conjugated lipids in BALB/c mice (n =
4). Blood samples were collected at 15 mins, 30 mins, 1 h, 3 h, and 6 h post-dose.

| | Mean Ionizable Lipid Concentration (ng/mL Plasma) ± Std Dev (ng/mL | | | | | |
|---|---|---|---|---|---|---|
| | Plasma) | | | | | |
| | PEG2000- | Compound | Compound | Compound | Compound | Compound |
| Time (h) | C-DMA | 11 | 12 | 13 | 14 | 15 |
| 0.25 | 5.5E+04 ± | 1.5E+04 ± | 1.9E+04 ± | 2.5E+04 ± | 5.0E+04 ± | 1.1E+04 ± |
| | 8.9E+03 | 3.5E+04 | 3.8E+03 | 4.1E+03 | 6.1E+03 | 2.6E+03 |
| 0.50 | 1.9E+04 ± | 1.5E+04 ± | 2.2E+04 ± | 2.0E+04 ± | 1.6E+04 ± | 1.3E+04 ± |
| | 4.1E+03 | 4.4E+03 | 4.6E+03 | 4.1E+03 | 2.0E+03 | 2.3E+03 |
| 1.00 | 9.9E+03 ± | 1.1E+04 ± | 1.8E+04 ± | 1.2E+04 ± | 1.1E+04 ± | 1.2E+04 ± |
| | 8.3E+02 | 2.7E+03 | 2.4E+03 | 3.9E+03 | 1.9E+03 | 1.2E+03 |
| 3.00 | 4.6E+03 ± | 5.6E+03 ± | 1.0E+04 ± | 7.8E+03 ± | 5.6E+03 ± | 6.4E+03 ± |
| | 1.5E+02 | 1.2E+03 | 6.3E+02 | 2.0E+03 | 1.2E+03 | 6.6E+02 |
| 6.00 | 2.7E+03 ± | 2.5E+03 ± | 5.9E+03 ± | 6.4E+03 ± | 2.5E+03 ± | 4.0E+03 ± |
| | 1.7E+02 | 4.0E+02 | 1.2E+03 | 5.6E+02 | 4.0E+02 | 4.4E+02 |

### Example 8. Effect of PEOZ-Lipid Ratio and Structure on Particle Characteristics

PEOZ lipids of ˜5000 Da with dialkyl C14 chains were formulated into LNPs at various molar ratios (0.5%, 1.5%, and 3.0%). Despite the hydrophobicity of the PEOZ 5000 polymer, its incorporation into LNP resulted in formation of stable particles, where size was dependent on the amount of PEOZ lipid. Particle size decreased with increasing amounts of PEOZ lipid (Table 6). With a fixed PEOZ lipid concentration of 1.5 mol %, varying the PEOZ lipid chemistry did not significantly impact particle characteristics (Table 7). Collectively, these results demonstrate that PEOZ formulations can be engineered through variation of chain length and molar ratio.

TABLE 6
Particle characteristics of Ovalbumin mRNA-LNP formulations
containing a titration of PEOZ5000-DMA lipid

| | | Final Product | |
|---|---|---|---|
| LNP mol ratios of polymer-conjugated | | Z-avg | % |
| lipid:Cationic lipid:Cholesterol:DSPC | | (PDI) nm | EE |
| PEG2000-C-DMA (2xC14) | 1.5:50.0:38.5:10.0 | 72 (0.06) | 98 |
| Compound 14 | 0.5:50.5:38.9:10.1 | 97 (0.05) | 96 |
| Compound 14 | 1.5:50.0:38.5:10.0 | 68 (0.20) | 95 |
| Compound 14 | 3.0:49.2:37.9:9.8 | 60 (0.10) | 89 |

% EE = encapsulation efficiency;
PDI = polydispersity

TABLE 7
Particle characteristics of Ovalbumin mRNA-LNP formulations
containing PEOZ lipids

| | Final Product | |
|---|---|---|
| LNP mol ratios of 1.5:50.0:38.5:10.0, | Z-avg | % |
| Polymer-conjugated lipid:Cationic lipid:Cholesterol:DSPC | (PDI) nm | EE |
| PEG2000-C-DMA (2xC14) | 72 (0.07) | 98 |
| Compound 14 | 67 (0.08) | 97 |
| Compound 11 | 65 (0.08) | 94 |
| Compound 12 | 71 (0.11) | 87 |
| Compound 13 | 72 (0.11) | 89 |
| Compound 15 | 68 (0.10) | 92 |

% EE = encapsulation efficiency;
PDI = polydispersity

### Example 9. Effect of Lipid Ratios on PEOZ-LNP Immunogenicity in Ovalbumin Model

LNP formulations containing varying amounts of PEOZ5000 lipids were assessed for their ability to induce antigen-specific immunogenicity following intramuscular (IM) administration in an ovalbumin (OVA) model. This model identifies the efficacy of formulations in a vaccine setting. Formulations were compared to the PEG-lipid benchmark controls.

Animals were treated with LNP in the prime/boost regimen described in Example 4, then assessed for levels of anti-OVA IgG antibody produced at Day 35. LNP containing 0.5% PEOZ produced equivalent levels of anti-OVA IgG compared to the benchmark control (Table 8). These trends followed through in the analysis for interferon-gamma producing splenocytes (Table 9). Alternate PEOZ-lipid structures were also tested, and resulted in comparable levels of anti-OVA IgG levels (Table 10), as well as similar or higher levels of interferon-gamma producing splenocytes (Table 11) than the benchmark.

These results demonstrate that LNP containing PEOZ-lipids can be used to deliver mRNA encoding antigens, generating a host immune response against those antigens, and therefore successfully incorporated into vaccine platforms. PEG is found in many other products, such as cosmetics, reportedly causing a small subset of the population to raise antibodies against PEG. This may cause adverse reactions when such individuals are exposed to PEG in the form of LNP, such as the coronavirus vaccines (Shimabukuro 2021). The ability to replace the PEG lipids in LNP with alternate polymer-conjugated lipids, such as PEOZ-lipids, therefore has great utility.

TABLE 8
Anti-OVA IgG levels on Day 35 following intramuscular
administration of OVA mRNA LNP at 1 μg dose
on Day 0 and Day 21 in BALB/c mice (n = 4)

| | | Day 35 | |
|---|---|---|---|
| | | Anti-OVA | |
| LNP mol ratios of polymer-conjugated | | IgG | Std Dev |
| lipid:Cationic lipid:Cholesterol:DSPC | | (ng/ml) | (ng/ml) |
| PEG2000-C-DMA (2xC14) | 1.5:50.0:38.5:10.0 | 135719 | 15373 |
| Compound 14 | 0.5:50.5:38.9:10.1 | 136188 | 36361 |
| Compound 14 | 1.5:50.0:38.5:10.0 | 69994 | 1540 |
| Compound 14 | 3.0:49.2:37.9:9.8 | 47318 | 18130 |

TABLE 9
IFN-gamma producing splenocytes on Day 35 following
intramuscular administration of OVA mRNA LNP at 1 μg
dose on Day 0 and Day 21 in BALB/c mice (n = 4)

| | | Day 35 | |
|---|---|---|---|
| | | | Std Dev |
| LNP mol ratios of polymer-conjugated | | SFU/10⁶ | (SFU/10⁶ |
| lipid:Cationic lipid:Cholesterol:DSPC | | splenocytes | splenocytes) |
| PEG2000-C-DMA | 1.5:50.0:38.5:10.0 | 328 | 150 |
| Compound 14 | 0.5:50.5:38.9:10.1 | 629 | 382 |
| Compound 14 | 1.5:50.0:38.5:10.0 | 266 | 142 |
| Compound 14 | 3.0:49.2:37.9:9.8 | 67 | 52 |

TABLE 10
Anti-OVA IgG levels on Day 35 following intramuscular
administration of OVA mRNA LNP at 1 μg dose
on Day 0 and Day 21 in BALB/c mice (n = 4)

| | Day 35 | |
|---|---|---|
| LNP mol ratios of 1.5:50.0:38.5:10.0, | Anti-OVA | Std |
| Polymer-conjugated lipid:Cationic | IgG | Dev |
| lipid:Cholesterol:DSPC | (ng/ml) | (ng/ml) |
| PEG2000-C-DMA (2xC14) | 74670 | 14235 |
| Compound 14 | 81284 | 32301 |
| Compound 11 | 73613 | 16774 |
| Compound 12 | 64818 | 28087 |
| Compound 13 | 85087 | 20689 |
| Compound 15 | 67821 | 35758 |

TABLE 11
IFN-gamma producing splenocytes on Day 35 following intramuscular administration
of OVA mRNA LNP at 1 μg dose on Day 0 and Day 21 in BALB/c mice (n = 4)

| | Day 35 | |
|---|---|---|
| | | Std Dev |
| LNP mol ratios of 1.5:50.0:38.5:10.0, | SFU/10⁶ | (SFU/10⁶ |
| Polymer-conjugated lipid:Cationic lipid:Cholesterol:DSPC | splenocytes | splenocytes) |
| PEG2000-C-DMA (2xC14) | 165 | 35 |
| Compound 14 | 226 | 103 |
| Compound 11 | 342 | 61 |
| Compound 12 | 320 | 185 |
| Compound 13 | 328 | 198 |
| Compound 15 | 219 | 76 |

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents, PCT publications, and Genbank Accession Nos., are incorporated herein by reference for all purposes.

What is claimed is:

1. A poly(alkyloxazoline)-lipid conjugate according to Formula I: 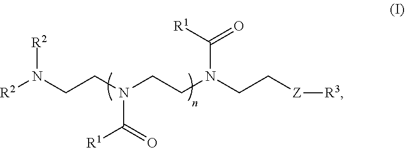
or a pharmaceutically acceptable salt thereof, wherein:
subscript n is an integer ranging from 10 to 100;
R¹ is independently C₁₋₆ alkyl;
each R² is independently selected from the group consisting of hydrogen, C₁₋₆ alkyl, and C₁₋₆ acyl; and
(i) Z is —S(CH₂)₂C(O)—, and
R³ is selected from the group consisting of —NR^{3a}R^{3b}, —NR^{3a}CH₂CH(R^{3b})₂, —CH(R^{3b})₂, and —CH₂CH(R^{3b})₂;
or
(ii) Z is selected from the group consisting of —Z¹—OC(O)—, —Z¹—NHC(O)—, —Z¹—S(O)₂—, and —Z¹—OCH₂—,
Z¹ is selected from the group consisting of a covalent bond, an oligo(ethylene glycol) diradical, and a poly(ethylene glycol) diradical, and
R³ is selected from the group consisting of —NR^{3a}R^{3b} and —CH(R^{3b})₂;
wherein each R^{3a} and R^{3b} is independently selected from the group consisting of hydrogen, C₆₋₂₂ alkyl, C₆₋₂₂ alkenyl, and C₆₋₂₂ alkynyl, wherein one or more non-adjacent CH₂ groups in R^{3a} and R^{3b} are optionally and independently replaced with oxygen,
provided that at least one R^{3a} or R^{3b} is selected from the group consisting of C₆₋₂₂ alkyl, C₆₋₂₂ alkenyl, and C₆₋₂₂ alkynyl, wherein one or more non-adjacent CH₂ groups in R^{3a} or R^{3b} are optionally and independently replaced with oxygen.

2. The poly(alkyloxazoline)-lipid conjugate of claim 1, or a pharmaceutically acceptable salt thereof, wherein R¹ is ethyl.

3. The poly(alkyloxazoline)-lipid conjugate of claim 1, or a pharmaceutically acceptable salt thereof, wherein R¹ is methyl.

4. The poly(alkyloxazoline)-lipid conjugate of claim 1, or a pharmaceutically acceptable salt thereof, wherein each R¹ is independently selected from the group consisting of ethyl and methyl.

5. The poly(alkyloxazoline)-lipid conjugate of any one of claims 1-4, or a pharmaceutically acceptable salt thereof, wherein subscript n is an integer ranging from about 15 to about 55.

6. The poly(alkyloxazoline)-lipid conjugate of claim 5, or a pharmaceutically acceptable salt thereof, wherein the poly(alkyloxazoline) portion of the conjugate has a number average molecular weight ranging from about 2,000 Da to about 5,000 Da.

7. The poly(alkyloxazoline)-lipid conjugate of any one of claims 1-6, having a structure according to Formula IIa: 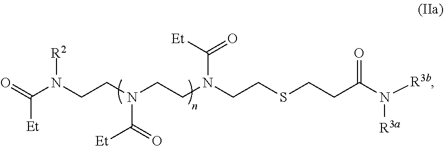
or a pharmaceutically acceptable salt thereof, wherein:
R² is selected from the group consisting of hydrogen and methyl;
R^{3a} is selected from the group consisting of hydrogen, C₁₂₋₁₈ alkyl, and C₁₂₋₁₈ alkenyl; and
R^{3b} is selected from the group consisting of C₁₂₋₁₈ alkyl and C₁₂₋₁₈ alkenyl.

8. The poly(alkyloxazoline)-lipid conjugate of claim 7, wherein R^{3a} and R^{3b} are independently selected from the group consisting of C₁₂₋₁₈ alkyl and C₁₂₋₁₈ alkenyl.

9. The poly(alkyloxazoline)-lipid conjugate of any one of claims 1-6, having a structure according to Formula IIb: 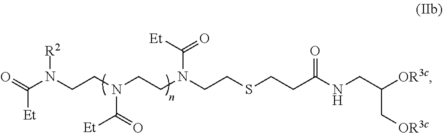
or a pharmaceutically acceptable salt thereof, wherein:
R² is selected from the group consisting of hydrogen and methyl; and
each R³, is independently selected from the group consisting of C₁₂₋₁₈ alkyl and C₁₂₋₁₈ alkenyl.

10. The poly(alkyloxazoline)-lipid conjugate of any one of claims 1-6, wherein Z is —Z¹—OC(O)—, Z¹ is selected from the group consisting of a covalent bond and a poly(ethylene glycol) diradical, and R³ is —NR^{3a}R^{3b}.

11. The poly(alkyloxazoline)-lipid conjugate of any one of claims 1-6, wherein Z is —Z¹—NHC(O)—, Z¹ is a covalent bond, and R³ is —NR^{3a}R^{3b}.

12. The poly(alkyloxazoline)-lipid conjugate of any one of claims 1-6, wherein Z is and R³ is —Z¹—OC(O)—, Z¹ is a covalent bond, and R³ is —CH(R^{3b})₂.

13. The poly(alkyloxazoline)-lipid conjugate of any one of claims 1-6, wherein Z is —Z¹—S(O)₂—, Z¹ is a covalent bond, and R³ is —NR^{3a}R^{3b}.

14. The poly(alkyloxazoline)-lipid conjugate of any one of claims 1-6, wherein Z is —Z¹—OCH₂—, Z¹ is a covalent bond, and R³ is —CH(R^{3b})₂.

15. The poly(alkyloxazoline)-lipid conjugate of any one of claims 1-6, wherein Z is —Z¹—NHC(O)—, Z¹ is a covalent bond, and R³ is —CH(R^{3b})₂.

16. The poly(alkyloxazoline)-lipid conjugate of any one of claims 10-15, wherein R^{3a} and each R^{3b} are independently selected from the group consisting of C₁₂₋₁₈ alkyl and C₁₂₋₁₈ alkenyl.

17. A lipid nanoparticle comprising a poly(alkyloxazoline)-lipid conjugate according to any one of claims 1-16.

18. The lipid nanoparticle of claim 17, further comprising a cationic lipid, a neutral lipid, a sterol, or a combination thereof.

19. The lipid nanoparticle of claim 17 or 18, further comprising a nucleic acid.

20. A lipid nanoparticle comprising:
(a) a nucleic acid;
(b) a cationic lipid comprising from 30 mol % to 80 mol % of the total lipid present in the lipid nanoparticle;
(c) a neutral lipid;
(d) a sterol; and
(e) a poly(alkyloxazoline)-lipid conjugate comprising from 0.1 mol % to 10 mol % of the total lipid present in the lipid nanoparticle.

21. The lipid nanoparticle of claim 20, wherein the cationic lipid comprises from 40 mol % to 70 mol % of the total lipid present in the lipid nanoparticle.

22. The lipid nanoparticle of claim 20 or 21, wherein the cationic lipid comprises from 45 mol % to 65 mol % of the total lipid present in the lipid nanoparticle.

23. The lipid nanoparticle of any one of claims 20-22, wherein the cationic lipid comprises from 45 mol % to 60 mol % of the total lipid present in the lipid nanoparticle.

24. The lipid nanoparticle of any one of claims 20-23, wherein the cationic lipid comprises from 50 mol % to 60 mol % of the total lipid present in the lipid nanoparticle.

25. The lipid nanoparticle of any one of claims 20-24, wherein the neutral lipid comprises from 3 mol % to 20 mol % of the total lipid present in the lipid nanoparticle.

26. The lipid nanoparticle of any one of claims 20-25, wherein the neutral lipid comprises from 5 mol % to 15 mol % of the total lipid present in the lipid nanoparticle.

27. The lipid nanoparticle of any one of claims 20-26, wherein the neutral lipid comprises from 8 mol % to 12 mol % of the total lipid present in the lipid nanoparticle.

28. The lipid nanoparticle of any one of claims 20-27, wherein the sterol comprises from 10 mol % to 60 mol % of the total lipid present in the lipid nanoparticle.

29. The lipid nanoparticle of any one of claims 20-28, wherein the sterol comprises from 20 mol % to 50 mol % of the total lipid present in the lipid nanoparticle.

30. The lipid nanoparticle of any one of claims 20-29, wherein the sterol comprises from 30 mol % to 40 mol % of the total lipid present in the lipid nanoparticle.

31. The lipid nanoparticle of any one of claims 20-30, wherein the poly(alkyloxazoline)-lipid conjugate comprises from 0.1 mol % to 5 mol % of the total lipid present in the lipid nanoparticle.

32. The lipid nanoparticle of any one of claims 20-31, wherein the poly(alkyloxazoline)-lipid conjugate comprises from 0.5 mol % to 3 mol % of the total lipid present in the lipid nanoparticle.

33. The lipid nanoparticle of claim 20, wherein the cationic lipid comprises from 50 mol % to 60 mol % of the total lipid present in the lipid nanoparticle, the neutral lipid comprises from 8 mol % to 12 mol % of the total lipid present in the lipid nanoparticle, the sterol comprises from 30 mol % to 40 mol % of the total lipid present in the lipid nanoparticle, and the poly(alkyloxazoline)-lipid conjugate comprises from 0.5 mol % to 3 mol % of the total lipid present in the lipid nanoparticle.

34. The lipid nanoparticle of any one of claims 20-33, wherein the neutral lipid comprises a phospholipid.

35. The lipid nanoparticle of any one of claims 20-34, wherein the sterol comprises cholesterol.

36. The lipid nanoparticle of any one of claims 20-35, wherein the poly(alkyloxazoline)-lipid conjugate comprises a poly(alkyloxazoline)-lipid conjugate according to any one of claims 1-16.

37. The lipid nanoparticle of any one of claims 19-35, wherein the nucleic acid comprises an RNA.

38. The lipid nanoparticle of claim 37, wherein the RNA comprises an mRNA.

39. The lipid nanoparticle of any one of claims 19-38, wherein the nucleic acid is fully encapsulated in the lipid nanoparticle.

40. The lipid nanoparticle of any one of claims 17-39, wherein the lipid nanoparticle has a mean diameter ranging from 40 nm to 150 nm.

41. A pharmaceutical composition comprising a lipid nanoparticle of any one of claims 17-40 and a pharmaceutically acceptable carrier.

42. The pharmaceutical composition of claim 41, wherein the pharmaceutical composition is formulated for intravenous, intramuscular, pulmonary, intracerebral, intrathecal, or intranasal administration.

43. A method for introducing a nucleic acid into a cell, the method comprising:
contacting the cell with a lipid nanoparticle of any one of claims 17-40 or a pharmaceutical composition of claim 41 or 42.

44. A method for delivering a nucleic acid to a subject, the method comprising:
administering to the subject a lipid nanoparticle of any one of claims 17-40 or a pharmaceutical composition of claim 41 or 42.

45. A method for preventing or treating a disease or disorder in a subject in need thereof, the method comprising:
administering to the subject a lipid nanoparticle of any one of claims 17-40 or a pharmaceutical composition of claim 41 or 42.

46. The method of claim 45, wherein the disease or disorder is a viral infection, a liver disease or disorder, a lung disease or disorder, a disease or disorder of the CNS, or cancer.
